(19) United States
(12) Patent Application Publication
(10) Pub. No.: US 2024/0052058 A1
(43) Pub. Date: Feb. 15, 2024

(54) METHODS OF USING A BISPECIFIC ANTIBODY THAT RECOGNIZES COAGULATION FACTOR IX AND/OR ACTIVATED COAGULATION FACTOR IX AND COAGULATION FACTOR X AND/OR ACTIVATED COAGULATION FACTOR X

(71) Applicants: Chugai Seiyaku Kabushiki Kaisha, Tokyo (JP); F. Hoffmann-La Roche AG, Basel (CH)

(72) Inventors: Koichiro Yoneyama, Tokyo (JP); Christophe Schmitt, Basel (CH)

(73) Assignees: Chugai Seiyaku Kabushiki Kaisha, Tokyo (JP); F. Hoffmann-La Roche AG, Basel (CH)

(21) Appl. No.: 18/466,900

(22) Filed: Sep. 14, 2023

Related U.S. Application Data
(62) Division of application No. 18/156,559, filed on Jan. 19, 2023, now abandoned.
Application No. 18/156,559 is a division of application No. 17/828,752, filed on May 31, 2022, now abandoned.
Application No. 17/828,752 is a division of application No. 16/330,269, filed on Mar. 4, 2019, now Pat. No. 11,352,438.
(60) Provisional application No. 62/485,514, filed on Apr. 14, 2017.
(60) Provisional application No. 62/437,281, filed on Dec. 21, 2016.
(60) Provisional application No. 62/383,933, filed on Sep. 6, 2016.

(51) Int. Cl. C07K 16/36; A61P 7/04

(52) U.S. Cl. CPC C07K 16/36; A61P 7/04; C07K 2317/24; C07K 2317/31; C07K 2317/94; A61K 2039/545

(57) ABSTRACT

An objective of the present invention is to provide an effective pharmaceutical composition or a dosage regimen for preventing and/or treating bleeding, a disease accompanying bleeding, or a disease caused by bleeding. The inventors discovered that by administering a pharmaceutical composition comprising a bispecific antigen-binding molecule that recognizes (a) blood coagulation factor IX and/or activated blood coagulation factor IX and (b) blood coagulation factor X and/or activated blood coagulation factor X according to a given dosage regimen, bleeding, a disease accompanying bleeding, or a disease caused by bleeding can be prevented and/or treated more effectively.

### CROSS REFERENCE TO RELATED APPLICATIONS

The present application is a divisional application of U.S. application Ser. No. 18/156,559, filed Jan. 19, 2023, which is a divisional application of U.S. application Ser. No. 17/828,752, filed May 31, 2022 (now abandoned), which is a divisional application of U.S. application Ser. No. 16/330,269, filed Mar. 4, 2019 (now U.S. Pat. No. 11,352,438), which is the National Stage of International Application No. PCT/JP2017/031933, filed Sep. 5, 2017, which claims the benefit of U.S. Provisional Application No. 62/383,933 filed Sep. 6, 2016, U.S. Provisional Application No. 62/437,281 filed Dec. 21, 2016, and U.S. Provisional Application No. 62/485,514 filed Apr. 14, 2017. The entire disclosures of these applications are hereby incorporated by reference.

### SEQUENCE LISTING

This application contains a Sequence Listing that has been submitted electronically as an XML file named C1-A1611P-JPD1sq.xml. The XML file, created on Aug. 25, 2023, is 21,573 bytes in size. The material in the XML file is hereby incorporated by reference in its entirety.

### TECHNICAL FIELD

The present invention relates generally to the field of therapeutics for bleeding disorders.

### BACKGROUND ART

Hemophilia is a hemorrhagic disease caused by a congenital deficiency or dysfunction of coagulation factor VIII (FVIII) or coagulation factor IX (FIX). The former is called hemophilia A and the latter is called hemophilia B. Genes for both factors are located on the X chromosome, and since genetic defects take the X-chromosome-linked recessive hereditary form, 99% or more of the patients who develop the disease are men. It is known that the prevalence rate is approximately one in 10,000 live male births, and the ratio between hemophilia A and hemophilia B is approximately 5:1.

The severity of hemophilia A is defined by the FVIII activity in blood. Patients with an activity of less than 1% are classified as severe, patients with an activity of 1% to 5% are classified as moderate, and patients with an activity of more than 5% and less than 40% are classified as mild. Severe patients who account for approximately half of hemophilia A patients exhibit bleeding symptoms several times a month, and this frequency is markedly high as compared to those of moderate and mild patients.

For bleeding in hemophilia A patients, FVIII formulations are generally administered on demand (on-demand therapy). In recent years, FVIII formulations are also administered prophylactically to prevent bleeding events (regular replacement therapy; NPLs 1 and 2). The half-life of FVIII formulations in blood is approximately 8 to 19 hours. Therefore, for continuous prevention, FVIII formulations are administered to patients three times a week (NPLs 3 and 4). In on- demand therapy, FVIII formulations are also additionally administered at regular intervals as necessary to prevent reoccurrence of bleeding. In addition, FVIII formulations are mainly administered at home, but since they are administered intravenously, the difficulty of securing a blood vessel is a problem. Therefore, there has been a strong need for pharmaceutical agents with a lesser burden regarding their administration as compared to FVIII formulations.

Occasionally, antibodies against FVIII (inhibitors) develop in hemophilia A patients. Such inhibitors counteract the effects of the FVIII formulations. For bleeding in patients who have developed inhibitors (inhibitor patients), bypassing agents are administered. Their mechanisms of action are not dependent on FVIII function, that is, on the function of catalyzing the activation of blood coagulation factor X (FX) by activated blood coagulation factor IX (FIXa). Therefore, in some cases, bypassing agents cannot sufficiently stop the bleeding. Recently, results suggesting the effectiveness of regular administration therapy of bypassing agents have been obtained, but this has not yielded a sufficient effect to suppress bleeding as compared to FVIII formulations. Accordingly, there has been a strong need for therapeutic agents that can be administered subcutaneously, as well as long-acting therapeutic agents that can be administered less frequently, regardless of the presence of inhibitors.

Recently, as a means for solving the problem, an antibody that functionally substitutes for FVIII, emicizumab (ACE910, RO5534262) and its use were disclosed (PTLs 1, 2, 3, 4, and 5, NPLs 5, 6, 7, and 8).

Emicizumab is a recombinant humanized bispecific antibody that binds to (a) FIX and/or FIXa and (b) FX and/or activated blood coagulation factor FX (FXa), and mimics the cofactor function of FVIII. Japanese patients with severe hemophilia A (with or without factor VIII inhibitors) were enrolled in an open-label, non-randomized, inter-individual dose-escalation study of emicizumab. Enrolled patients were assigned to cohort 1, cohort 2, or cohort 3, and received subcutaneous emicizumab at an initial dose of 1.0 mg per kilogram of body weight (cohort 1) or 3 mg per kilogram (cohorts 2 and 3) at week 0 (day 1), followed by a once-weekly subcutaneous dose of 0.3, 1.0, or 3 mg per kilogram (cohorts 1, 2, and 3, respectively) from week 1 through week 12. The initial and subsequent doses for cohort 3 were the same. Emicizumab decreased the annualized bleeding rates (ABR) in patients who had hemophilia A with or without factor VIII inhibitors (NPL 9). At the completion of the 12-week study, eligible patients were able to participate in an extension study (NPL 10).

### CITATION LIST

### Patent Literature

[PTL 1] WO 2005/035754
[PTL 2] WO 2005/035756
[PTL 3] WO 2006/109592
[PTL 4] WO 2012/067176
[PTL 5] WO 2015/194233

### Non-Patent Literature

[NPL 1] Blood 58, 1-13 (1981)
[NPL 2] Nature 312, 330-337 (1984)
[NPL 3] Nature 312, 337-342 (1984)
[NPL 4] Biochim.Biophys.Acta 871, 268-278 (1986)
[NPL 5] Nature Medicine 18, 1570-1574(2012)
[NPL 6] PLOS ONE 8, 1-13(2013)
[NPL 7] J Thromb Haemost. 12, 206-213(2014)
[NPL 8] Blood 127(13), 1633-1641(2016)
[NPL 9] N Engl J Med. 374(21), 2044-2053(2016)
[NPL 10] XXV Congress of the International Society on Thrombosis and Haemostasis, Toronto, Canada, Jun. 20-25, 2015. Abstr AS017.

### SUMMARY OF INVENTION

### Technical Problem

An objective of the present invention is to provide another effective pharmaceutical composition or a dosage regimen for treating and/or reducing the incidence of a disease that develops and/or progresses due to a decrease or deficiency in the activity of FVIII and/or activated blood coagulation factor VIII (FVIIIa).

### Solution to Problem

As a result of dedicated research, the present inventors succeeded in discovering an effective dosage regimen for a pharmaceutical composition containing a bispecific antigen-binding molecule (antibody) that recognizes (a) FIX and/or FIXa and (b) FX and/or FXa for treating and/or reducing the incidence of a disease that develops and/or progresses due to a decrease or deficiency in the activity of FVIII and/or FVIIIa.

Specifically, the present invention relates to a pharmaceutical composition or a dosage regimen used for treating and/or reducing the incidence of a disease that develops and/or progresses due to a decrease or deficiency in the activity of FVIII and/or FVIIIa, and specifically relates to the following:
[1] a method for treating and/or reducing the incidence of a disease that develops and/or progresses due to a decrease or deficiency in the activity of blood coagulation factor VIII and/or activated blood coagulation factor VIII (FVIIIa), the method comprising: administering to a subject a bispecific antibody that recognizes (a) blood coagulation factor IX and/or activated blood coagulation factor IX and (b) blood coagulation factor X and/or activated blood coagulation factor X at a weekly loading dose of 3 mg/kg or 4.5 mg/kg of the antibody for one or more weeks or at an every-two-week loading dose of 6 mg/kg of the antibody for two or more weeks; and, after the loading dose administration(s) are complete, administering a maintenance dose of the antibody to the subject one or more times, the maintenance dose being 6 mg/kg of the antibody;
[2] the method of [1], wherein the maintenance dose of 6 mg/kg of the antibody is administered to the subject every four weeks or every month in a single dose or multiple divided doses;
[3] the method of [1] or [2], wherein the antibody is administered at the weekly loading dose of 3 mg/kg of the antibody for four weeks, followed by the maintenance dose;
[4] the method of [1] or [2], wherein the antibody is administered at the weekly loading dose of 4.5 mg/kg of the antibody for two weeks, followed by the maintenance dose;
[5] the method of [1] or [2], wherein the antibody is administered at the every-two-week loading dose of 6 mg/kg of the antibody for four weeks, followed by the maintenance dose;
[6] the method of any one of [1] to [5], wherein the maintenance dose is administered in one single dose at 6 mg/kg of the antibody monthly or every four weeks;
[7] the method of any one of [1] to [5], wherein the maintenance dose is administered in two single doses of the antibody, each at 3 mg/kg, monthly or every four weeks, wherein one single dose of the maintenance dose at 3 mg/kg of the antibody is administered once every two weeks;
[8] the method of any one of [1] to [5], wherein the maintenance dose is administered in four single doses each at 1.5 mg/kg of the antibody monthly or every four weeks, wherein one single dose of the maintenance dose at 1.5 mg/kg of the antibody is administered once every week;
[9] the method of any one of [1] to [8] further comprising, in a case of no or insufficient effect of treating and/or reducing the incidence of the disease by administering the maintenance dose of the antibody, stopping administering the maintenance dose of the antibody and starting administering an alternative maintenance dose of the antibody to the subject, the alternative maintenance dose being a weekly dose of 3 mg/kg of the antibody or an every-two-week dose of 6 mg/kg of the antibody;
[10] a method for treating and/or reducing the incidence of a disease that develops and/or progresses due to a decrease or deficiency in the activity of blood coagulation factor VIII and/or activated blood coagulation factor VIII (FVIIIa), the method comprising: administering to a subject a bispecific antibody that recognizes (a) blood coagulation factor IX and/or activated blood coagulation factor IX and (b) blood coagulation factor X and/or activated blood coagulation factor X at a weekly loading dose of 4.5 mg/kg of the antibody for four weeks; and thereafter administering a maintenance dose of the antibody to the subject one or more times, the maintenance dose being 9 mg/kg of the antibody which is administered every four weeks or every month in two or four divided doses;
[11] a method for treating and/or reducing the incidence of a disease that develops and/or progresses due to a decrease or deficiency in the activity of blood coagulation factor VIII and/or activated blood coagulation factor VIII (FVIIIa), the method comprising: administering to a subject a bispecific antibody that recognizes (a) blood coagulation factor IX and/or activated blood coagulation factor IX and (b) blood coagulation factor X and/or activated blood coagulation factor X at a weekly loading dose of 6 mg/kg of the antibody for four weeks; and thereafter administering a maintenance dose of the antibody to the subject one or more times, the maintenance dose being 12 mg/kg of the antibody which is administered every four weeks or every month in two or four divided doses;
[12] the method of [10] further comprising, in a case of no or insufficient effect of treating and/or reducing the incidence of the disease by administering the maintenance dose of the antibody, stopping administering the maintenance dose of the antibody and starting administering an alternative maintenance dose of the antibody to the subject, the alternative maintenance dose being a weekly dose of 4.5 mg/kg of the antibody;
[13] the method of [11] further comprising, in a case of no or insufficient effect of treating and/or reducing the incidence of the disease by administering the maintenance dose of the antibody, stopping administering the maintenance dose of the antibody and starting administering an alternative maintenance dose of the antibody to the subject, the alternative maintenance dose being a weekly dose of 6 mg/kg of the antibody;
[14] the method of any one of [1] to [13], wherein the antibody is emicizumab; and
[15] the method of [14], wherein the disease that develops and/or progresses due to a decrease or deficiency in the activity of blood coagulation factor VIII and/or activated blood coagulation factor VIII is selected from the group consisting of hemophilia A, acquired hemophilia A, von Willebrand disease, and hemophilia A with emergence of an inhibitor against blood coagulation factor VIII and/or activated blood coagulation factor VIII.

Furthermore, the present invention relates to:
[16] a pharmaceutical composition for use in treating and/or reducing the incidence of a disease that develops and/or progresses due to a decrease or deficiency in the activity of blood coagulation factor VIII and/or activated blood coagulation factor VIII (FVIIIa), wherein the composition comprises a bispecific antibody that recognizes (a) blood coagulation factor IX and/or activated blood coagulation factor IX and (b) blood coagulation factor X and/or activated blood coagulation factor X, wherein the bispecific antibody is administered at a weekly loading dose of 3 mg/kg or 4.5 mg/kg of the antibody for one or more weeks or at an every-two-week loading dose of 6 mg/kg of the antibody for two or more weeks and, after the loading dose administration(s) are complete, is administered at a maintenance dose one or more times, the maintenance dose being 6 mg/kg of the antibody;
[17] a pharmaceutical composition for use in treating and/or reducing the incidence of a disease that develops and/or progresses due to a decrease or deficiency in the activity of blood coagulation factor VIII and/or activated blood coagulation factor VIII (FVIIIa), wherein the composition comprises a bispecific antibody that recognizes (a) blood coagulation factor IX and/or activated blood coagulation factor IX and (b) blood coagulation factor X and/or activated blood coagulation factor X, wherein the bispecific antibody is administered at a weekly loading dose of 4.5 mg/kg of the antibody for four weeks and thereafter is administered at a maintenance dose one or more times, the maintenance dose being 9 mg/kg of the antibody which is administered every four weeks or every month in two or four divided doses;
[18] a pharmaceutical composition for use in treating and/or reducing the incidence of a disease that develops and/or progresses due to a decrease or deficiency in the activity of blood coagulation factor VIII and/or activated blood coagulation factor VIII (FVIIIa), wherein the composition comprises a bispecific antibody that recognizes (a) blood coagulation factor IX and/or activated blood coagulation factor IX and (b) blood coagulation factor X and/or activated blood coagulation factor X, wherein the bispecific antibody is administered at a weekly loading dose of 6 mg/kg of the antibody for four weeks and thereafter is administered at a maintenance dose one or more times, the maintenance dose being 12 mg/kg of the antibody which is administered every four weeks or every month in two or four divided doses;
[19] a product comprising (i) a container; (ii) a pharmaceutical composition in the container, wherein the composition comprises a bispecific antibody that recognizes (a) blood coagulation factor IX and/or activated blood coagulation factor IX and (b) blood coagulation factor X and/or activated blood coagulation factor X; and (iii) a document instructing (a) administration of the bispecific antibody at a weekly loading dose of 3 mg/kg or 4.5 mg/kg of the antibody for one or more weeks or at an every-two-week loading dose of 6 mg/kg of the antibody for two or more weeks and (b) one or more administration(s) of a maintenance dose of the bispecific antibody after the loading dose administration(s) are complete, the maintenance dose being 6 mg/kg of the antibody;
[20] a product comprising (i) a container; (ii) a pharmaceutical composition in the container, wherein the composition comprises a bispecific antibody that recognizes (a) blood coagulation factor IX and/or activated blood coagulation factor IX and (b) blood coagulation factor X and/or activated blood coagulation factor X; and (iii) a document instructing (a) administration of the bispecific antibody at a weekly loading dose of 4.5 mg/kg of the antibody for four weeks and (b) one or more administrations of a maintenance dose of the bispecific antibody after the loading dose administration(s) are complete, the maintenance dose being 9 mg/kg of the antibody which is administered every four weeks or every month in two or four divided doses;
[21] a product comprising (i) a container; (ii) a pharmaceutical composition in the container, wherein the composition comprises a bispecific antibody that recognizes (a) blood coagulation factor IX and/or activated blood coagulation factor IX and (b) blood coagulation factor X and/or activated blood coagulation factor X; and (iii) a document instructing (a) administration of the bispecific antibody at a weekly loading dose of 6 mg/kg of the antibody for four weeks and (b) one or more administrations of a maintenance dose of the bispecific antibody after the loading dose administration(s) are complete, the maintenance dose being 12 mg/kg of the antibody which is administered every four weeks or every month in two or four divided doses;
[22] a bispecific antibody for use in treating and/or reducing the incidence of a disease that develops and/or progresses due to a decrease or deficiency in the activity of blood coagulation factor VIII and/or activated blood coagulation factor VIII (FVIIIa), wherein the bispecific antibody recognizes (a) blood coagulation factor IX and/or activated blood coagulation factor IX and (b) blood coagulation factor X and/or activated blood coagulation factor X and is administered at a weekly loading dose of 3 mg/kg or 4.5 mg/kg of the antibody for one or more weeks or at an every-two-week loading dose of 6 mg/kg of the antibody for two or more weeks and, after the loading dose administration(s) are complete, is administered at a maintenance dose one or more times, the maintenance dose being 6 mg/kg of the antibody;
[23] a bispecific antibody for use in treating and/or reducing the incidence of a disease that develops and/or progresses due to a decrease or deficiency in the activity of blood coagulation factor VIII and/or activated blood coagulation factor VIII (FVIIIa), wherein the bispecific antibody recognizes (a) blood coagulation factor IX and/or activated blood coagulation factor IX and (b) blood coagulation factor X and/or activated blood coagulation factor X and is administered at a weekly loading dose of 4.5 mg/kg of the antibody for four weeks and thereafter is administered at a maintenance dose one or more times, the maintenance dose being 9 mg/kg of the antibody which is administered every four weeks or every month in two or four divided doses;
[24] a bispecific antibody for use in treating and/or reducing the incidence of a disease that develops and/or progresses due to a decrease or deficiency in the activity of blood coagulation factor VIII and/or activated blood coagulation factor VIII (FVIIIa), wherein the bispecific antibody recognizes (a) blood coagulation factor IX and/or activated blood coagulation factor IX and (b) blood coagulation factor X and/or activated blood coagulation factor X and is administered at a weekly loading dose of 6 mg/kg of the antibody for four weeks and thereafter is administered a maintenance dose one or more times, the maintenance dose being 12 mg/kg of the antibody which is administered every four weeks or every month in two or four divided doses;
[25] use of a bispecific antibody in the manufacture of a pharmaceutical composition used for treating and/or reducing the incidence of a disease that develops and/or progresses due to a decrease or deficiency in the activity of blood coagulation factor VIII and/or activated blood coagulation factor VIII (FVIIIa), wherein the bispecific antibody recognizes (a) blood coagulation factor IX and/or activated blood coagulation factor IX and (b) blood coagulation factor X and/or activated blood coagulation factor X, and is administered at a weekly loading dose of 3 mg/kg or 4.5 mg/kg of the antibody for one or more weeks or at an every-two-week loading dose of 6 mg/kg of the antibody for two or more weeks and, after the loading dose administration(s) are complete, is administered at a maintenance dose one or more times, the maintenance dose being 6 mg/kg of the antibody;
[26] use of a bispecific antibody in the manufacture of a pharmaceutical composition used for treating and/or reducing the incidence of a disease that develops and/or progresses due to a decrease or deficiency in the activity of blood coagulation factor VIII and/or activated blood coagulation factor VIII (FVIIIa), wherein the bispecific antibody recognizes (a) blood coagulation factor IX and/or activated blood coagulation factor IX and (b) blood coagulation factor X and/or activated blood coagulation factor X, and is administered at a weekly loading dose of 4.5 mg/kg of the antibody for four weeks and thereafter is administered at a maintenance dose one or more times, the maintenance dose being 9 mg/kg of the antibody which is administered every four weeks or every month in two or four divided doses; and
[27] use of a bispecific antibody in the manufacture of a pharmaceutical composition used for treating and/or reducing the incidence of a disease that develops and/or progresses due to a decrease or deficiency in the activity of blood coagulation factor VIII and/or activated blood coagulation factor VIII (FVIIIa), wherein the bispecific antibody recognizes (a) blood coagulation factor IX and/or activated blood coagulation factor IX and (b) blood coagulation factor X and/or activated blood coagulation factor X, and is administered at a weekly loading dose of 6 mg/kg of the antibody for four weeks and thereafter is administered at a maintenance dose one or more times, the maintenance dose being 12 mg/kg of the antibody which is administered every four weeks or every month in two or four divided doses.

### BRIEF DESCRIPTION OF DRAWINGS

FIG. 1 shows the simulated annual bleeding rate over plasma emicizumab concentrations. X-axis shows plasma emicizumab concentration (micro g/mL). Y-axis shows cumulative bleeding count for 52 weeks. Circles and solid line show the simulated medians plotted at each plasma emicizumab concentration. Shaded area shows the simulated 5th- to 95th-percentile range.

FIG. 2 shows the simulated change in plasma emicizumab concentration over time (weekly dosing). X-axis shows time (week) after first emicizumab administration. Y-axis shows plasma emicizumab concentration (micro g/mL). Circles and solid line show the simulated medians plotted at each trough sampling time point. Shaded area shows the simulated 5th- to 95th-percentile range. Broken line shows the target exposure level of 45 micro g/mL. Once-weekly loading dose of 3 mg/kg for first 4 weeks followed by once-weekly maintenance dose of 1.5 mg/kg was applied.

FIG. 3 shows the simulated change in plasma emicizumab concentration over time (two-weekly dosing). X-axis shows time (week) after first emicizumab administration. Y-axis shows plasma emicizumab concentration (micro g/mL). Circles and solid line show the simulated medians plotted at each trough sampling time point. Shaded area shows the simulated 5th- to 95th-percentile range. Broken line shows the target exposure level of 45 micro g/mL. Once-weekly loading dose of 3 mg/kg for first 4 weeks followed by once-every-two-week maintenance dose of 3 mg/kg was applied.

FIG. 4 shows the simulated change in plasma emicizumab concentration over time (four-weekly dosing). X-axis shows time (week) after first emicizumab administration. Y-axis shows plasma emicizumab concentration (micro g/mL). Circles and solid line show the simulated medians plotted at each trough sampling time point. Shaded area shows the simulated 5th- to 95th-percentile range. Broken line shows the target exposure level of 45 micro g/mL. Once-weekly loading dose of 3 mg/kg for first 4 weeks followed by once-every-four-week maintenance dose of 6 mg/kg was applied.

FIG. 5 shows the simulated distribution of annual bleeding rate (weekly dosing). X-axis shows cumulative bleeding count for 52 weeks. Y-axis shows proportion of patients having each cumulative bleeding count for 52 weeks (%). Bars show the simulated proportions. Once-weekly loading dose of 3 mg/kg for first 4 weeks followed by once-weekly maintenance dose of 1.5 mg/kg was applied.

FIG. 6 shows the simulated distribution of annual bleeding rate (two-weekly dosing). X-axis shows cumulative bleeding count for 52 weeks. Y-axis shows proportion of patients having each cumulative bleeding count for 52 weeks (%). Bars show the simulated proportions. Once-weekly loading dose of 3 mg/kg for first 4 weeks followed by once-every-two-week maintenance dose of 3 mg/kg was applied.

FIG. 7 shows the simulated distribution of annual bleeding rate (four-weekly dosing). X-axis shows cumulative bleeding count for 52 weeks. Y-axis shows proportion of patients having each cumulative bleeding count for 52 weeks (%). Bars show the simulated proportions. Once-weekly loading dose of 3 mg/kg for first 4 weeks followed by once-every-four-week maintenance dose of 6 mg/kg was applied.

FIG. 8 shows the annualized bleeding rates for study Arms A, B, and C in HAVEN 1 (Example 2). ABR, annualized bleeding rate; BPA, bypassing agent; RR, relative risk.

FIG. 9 shows the intra-participant comparison for treated bleeds in participants who received emicizumab prophylaxis (Arm C). Treated bleeds during the period when a participant received emicizumab prophylaxis are compared with treated bleeds during the period when the same participant had received previous prophylactic BPA treatment in 24 weeks prior to study entry in HAVEN 1.

FIG. 10 shows the observed trough emicizumab concentrations over time with once-weekly dosing (once-weekly loading dose of 3 mg/kg for first 4 weeks followed by once-weekly maintenance dose of 1.5 mg/kg) in HAVEN 1. SD, standard deviation.

FIG. 11 shows the intra-participant comparison for treated bleeds in participants who received emicizumab prophylaxis (Arm A). Treated bleeds during the period when a participant received emicizumab prophylaxis are compared with treated bleeds during the period when the same participant had received previous episodic BPA treatment in 24 weeks prior to study entry in HAVEN 1.

FIG. 12 shows the individual patient-calculated ABR for treated bleeds in HAVEN 2. The intra-participant comparison included patients previously enrolled in the NIS who were enrolled in HAVEN 2 for at least 12 weeks. Seven patients received prophylactic treatment with bypassing agents prior to the study, one received episodic treatment with bypassing agents. ABR, annualized bleeding rate; BPA, bypassing agent; NIS, non-interventional study.

FIG. 13 shows the observed and predicted emicizumab concentrations over time with once-four-weekly dosing in the PK Run-in Part of HAVEN 4 (Example 5). Emicizumab was administered subcutaneously at 6 mg/kg once every 4 weeks. Black dots and solid lines show the individual observations. Gray broken line shows the predicted mean. Gray bold solid lines indicate the upper and lower limits of the 95% prediction interval.

### DESCRIPTION OF EMBODIMENTS

A bispecific antigen-binding molecule that recognizes (a) blood coagulation factor IX (FIX) and/or activated blood coagulation factor IX (FIXa) and (b) blood coagulation factor X (FX) and/or activated blood coagulation factor X (FXa) preferably has an activity of functionally substituting for coagulation factor VIII (FVIII).

In the present invention, the phrase “functionally substitute for FVIII” means that (a) FIX and/or FIXa, and (b) FX and/or FXa are recognized, and the activation of FX by FIXa is promoted (FXa generation by FIXa is promoted). FXa generation-promoting activity can be evaluated using, for example, a measurement system comprising FIXa, FX, the synthetic substrate S-2222 (a synthetic substrate of FXa), and phospholipids. Such a measurement system shows the correlation between the severity of the disease and the clinical symptoms in hemophilia A cases (Rosen S, Andersson M, Blomback M et al. Clinical applications of a chromogenic substrate method for determination of FVIII activity. Thromb Haemost 1985; 54: 811-23).

Such antigen-binding molecules (such as antibodies) recognizing (a) FIX and/or FIXa and (b) FX and/or FXa can be obtained according to methods described, for example, in WO2005/035756, WO2006/109592, and WO2012/067176. Specifically, based on the sequences of antibodies against FIX and/or FIXa and antibodies against FX and/or FXa, antibodies can be generated using genetic recombination techniques known to those skilled in the art. Polynucleotide(s) encoding an antibody can be constructed based on the sequences of the antibodies against FIX and/or FIXa and antibodies against FX and/or FXa, and this can be inserted into an expression vector and subsequently expressed in appropriate host cells (see for example, Co, M. S. et al., J. Immunol. (1994) 152, 2968-2976; Better, M. and Horwitz, A. H., Methods Enzymol. (1989) 178, 476-496; Pluckthun, A. and Skerra, A., Methods Enzymol. (1989) 178, 497-515; Lamoyi, E., Methods Enzymol. (1986) 121, 652-663; Rousseaux, J. et al., Methods Enzymol. (1986) 121, 663-669; and Bird, R. E. and Walker, B. W., Trends Biotechnol. (1991) 9, 132-137).

In the present invention, the phrases “functionally substitute for FVIII” and “functionally substitute for FVIIIa” are used interchangeably.

Such bispecific antigen-binding molecules can be isolated from inside host cells or from outside the cells (such as from the medium), and can be purified as substantially pure and homogeneous antibodies. Isolation and purification of antibodies can be carried out using methods generally used for isolating and purifying antibodies. The methods are not limited and, for example, antibodies can be isolated and purified by appropriately selecting and combining column chromatography columns, filters, ultrafiltration, salting-out, solvent precipitation, solvent extraction, distillation, immunoprecipitation, SDS-polyacrylamide gel electrophoresis, isoelectric focusing, dialysis, recrystallization, and such.

The bispecific antigen-binding molecules of the present invention include the antibodies described, for example, in WO2005/035756, WO2006/109592, and WO2012/067176.

A bispecific antigen-binding molecule comprises a first antigen-binding site and a second antigen-binding site which can specifically bind to at least two different types of antigens. While the first antigen-binding site and the second antigen-binding site of the bispecific antigen-binding molecule of the present invention are not particularly limited as long as they have an activity to bind to (a) FIX and/or FIXa, and (b) FX and/or FXa, respectively, examples include sites necessary for binding with antigens, such as antibodies, scaffold molecules (antibody-like molecules), and peptides, and fragments containing such sites. A scaffold molecule is a molecule that exhibits a function by binding to a target molecule, and any polypeptide may be used as long as it is a conformationally stable polypeptide that can bind to at least one target antigen. Examples of such polypeptides include antibody variable regions, fibronectin (WO 2002/032925), protein A domain (WO 1995/001937), LDL receptor A domain (WO 2004/044011, WO 2005/040229), ankyrin (WO 2002/020565), as well as the molecules described in Nygren et al. (Current Opinion in Structural Biology, 7: 463-469 (1997); and Journal of Immunol Methods, 290: 3-28 (2004)), Binz et al. (Nature Biotech 23: 1257-1266 (2005)), and Hosse et al. (Protein Science 15: 14-27(2006)). Furthermore, as described in Curr Opin Mol Ther. 2010 August; 12(4): 487-95 and Drugs. 2008; 68(7): 901-12, peptide molecules that can bind to the target antigens can also be used.

Bispecific antigen-binding molecules can be produced using, for example, genetic recombination techniques (see, for example, Borrebaeck C A K and Larrick J W, THERAPEUTIC MONOCLONAL ANTIBODIES, Published in the United Kingdom by MACMILLAN PUBLISHERS LTD, 1990). Recombinant antibodies can be obtained by cloning DNAs encoding the antibodies from hybridomas or antibody-producing cells, such as sensitized lymphocytes that produce antibodies, inserting them into suitable vectors, and then introducing them into hosts (host cells) to produce the antibodies.

Furthermore, bispecific antigen-binding molecules may be whole antibodies, and may also be antibody fragments and low-molecular-weight antibodies, and modified antibodies.

For example, antibody fragments and low-molecular-weight antibodies include diabodies (Dbs), linear antibodies, and single chain antibody (hereinafter, also denoted as scFv) molecules. Herein, an “Fv” fragment is a smallest antibody fragment and comprises a full antigen recognition site and binding site.

Bispecific antibodies include human antibodies, mouse antibodies, rat antibodies, and such, and their origins are not limited. They may also be genetically modified antibodies, such as chimeric antibodies and humanized antibodies.

Methods for obtaining human antibodies are already known. For example, a human antibody of interest can be obtained by immunizing a transgenic animal carrying the entire repertoire of human antibody genes with an antigen of interest (see International Publication No. WO 93/12227, WO 92/03918, WO 94/02602, WO 94/25585, WO 96/34096, and WO 96/33735).

Genetically modified antibodies can also be produced using known methods. Specifically, for example, a chimeric antibody is an antibody that comprises H chain and L chain variable regions of an immunized animal antibody, and H chain and L chain constant regions of a human antibody. Chimeric antibodies can be obtained by linking DNAs encoding the variable regions of an antibody derived from an immunized animal with DNAs encoding the constant regions of a human antibody, inserting this into an expression vector, and then introducing this into host cells to produce the antibodies.

A humanized antibody is a modified antibody which is also often referred to as a reshaped human antibody. A humanized antibody is constructed by transferring the CDRs of an antibody derived from an immunized animal to the complementarity determining regions of a human antibody. General genetic recombination techniques for producing them are also known (see European Patent Application Publication No. EP 239400; International Publication No. WO 96/02576; Sato K et al., Cancer Research 1993, 53: 851-856; International Publication No. WO 99/51743).

More specifically, the bispecific antigen-binding molecule of the present invention is a bispecific antibody in which a first polypeptide and a third polypeptide are associated and a second polypeptide and a fourth polypeptide are associated, and is preferably emicizumab (ACE910, RO5534262) described below:
(a) a bispecific antibody comprising a first polypeptide which is an H chain containing an H chain variable region containing CDR 1, 2, and 3 amino acid sequences of SEQ ID NOs: 1, 2, and 3, respectively; a second polypeptide which is an H chain containing an H chain variable region containing CDR 1, 2, and 3 amino acid sequences of SEQ ID NOs: 6, 7, and 8, respectively; and a third and fourth polypeptide which are a commonly shared L chain containing an L chain variable region containing CDR 1, 2, and 3 amino acid sequences of SEQ ID NOs: 11, 12, and 13, respectively;
(b) a bispecific antibody comprising a first polypeptide which is an H chain containing an H chain variable region amino acid sequence of SEQ ID NO: 4;
a second polypeptide which is an H chain containing an H chain variable region amino acid sequence of SEQ ID NO: 9; and a third and fourth polypeptide which are a commonly shared L chain containing an L chain variable region amino acid sequence of SEQ ID NO: 14; or
(c) a bispecific antibody comprising a first polypeptide which is an H chain containing the amino acid sequence of SEQ ID NO: 5; a second polypeptide which is an H chain containing the amino acid sequence of SEQ ID NO: 10; and a third and fourth polypeptide which are a commonly shared L chain containing the amino acid sequence of SEQ ID NO: 15 (Q499-z121/J327-z119/L404-k).
Pharmaceutical compositions of the present invention which are used for therapeutic or preventive purposes can be prepared by mixing a therapeutic agent, if necessary, with suitable pharmaceutically acceptable carriers, vehicles, and such and made into a freeze-dry formulation or a solution formulation.
A “therapeutic agent” herein refers to the bispecific antigen-binding molecules of the present invention.
Examples of suitable pharmaceutically acceptable carriers and vehicles include sterilized water, physiological saline, stabilizers, excipients, antioxidants (such as ascorbic acid), buffers (such as phosphate, citrate, histidine, and other organic acids), antiseptics, surfactants (such as PEG and Tween), chelating agents (such as EDTA), and binders. They may also contain other low-molecular-weight polypeptides, proteins such as serum albumin, gelatin, and immunoglobulins, amino acids such as glycine, glutamine, asparagine, glutamic acid, aspartic acid, methionine, arginine, and lysine, sugars and carbohydrates such as polysaccharides and monosaccharides, and sugar alcohols such as mannitol and sorbitol. When preparing an aqueous solution for injection, for example, physiological saline and isotonic solutions containing glucose and other adjuvants such as D-sorbitol, D-mannose, D-mannitol, and sodium chloride may be used, and appropriate solubilizers such as alcohol (for example, ethanol), polyalcohols (such as propylene glycol and PEG), and nonionic surfactants (such as polysorbate 80, polysorbate 20, poloxamer 188, and HCO-50) may be used in combination. By mixing hyaluronidase into the formulation, a larger fluid volume can be administered subcutaneously (Expert Opin Drug Deliv. 2007 July; 4(4): 427-40). Further, the pharmaceutical compositions of the present invention may be preliminarily loaded in a syringe. Meanwhile, the solution formulation can be prepared according to the method described in WO 2011/090088.

If necessary, the antigen-binding molecules of the present invention can be encapsulated in microcapsules (e.g., those made of hydroxymethylcellulose, gelatin, and poly(methylmetacrylate)), or prepared as colloidal drug delivery systems (e.g., liposomes, albumin microspheres, microemulsion, nanoparticles, and nanocapsules) (see, for example, “Remington's Pharmaceutical Science 16th edition”, Oslo Ed. (1980)). Methods for preparing the pharmaceutical agents as controlled-release pharmaceutical agents are also well known, and such methods may be applied to the antigen-binding molecules of the present invention (Langer et al., J. Biomed. Mater. Res. 15: 267-277 (1981); Langer, Chemtech. 12: 98-105 (1982); U.S. Pat. No. 3,773,919; European Patent Application Publication No. EP 58,481; Sidman et al., Biopolymers 22: 547-556 (1983); EP 133,988).

A preferable liquid formulation is as follows.
20 mg/ml to 180 mg/ml emicizumab,
0.2 mg/ml to 1 mg/ml of poloxamer 188,
10 mM to 40 mM of histidine-aspartic acid buffer,
100 mM to 300 mM of arginine, at a pH of about 4.5 to 6.5.

The pharmaceutical compositions of the present invention can be administered to a patient via any appropriate route, for example, intravenously by bolus injection or continuous infusion for a given period, intramuscularly, intraperitoneally, intracerebrospinally, transdermally, subcutaneously, intraarticularly, sublingually, intrasynovially, orally, by inhalation, locally, or externally. Intravenous administration or subcutaneous administration is preferred.

The term “4 weeks” or “a month” as used herein are used interchangeably and the term “every 4 weeks”, “4-weekly”, “every month”, or “monthly” as used herein are used interchangeably.

The term “every 2 weeks”, “2-weekly”, or “bi-weekly” as used herein are used interchangeably.

A “maintenance” dose herein refers to one or more doses of a therapeutic agent administered to the patient over a treatment period. Different maintenance doses and different administration intervals can be combined.

In one aspect, the maintenance dose is between 0.3 mg/kg to 24 mg/kg of the antibody.

In one aspect, the administration interval is between one week to 24 weeks or 6 months.

In one aspect, the maintenance dose is 6 mg/kg of the antibody. The maintenance dose of 6 mg/kg refers to, for example, administering total 6 mg/kg of the antibody during 4 weeks or every month in a single dose or multiple divided doses.

In certain embodiment, the maintenance dose is 6 mg/kg of the antibody and this is administered in a single dose and the administration interval is 4 weeks (every month). In this case one single dose is administered during a month or 4 weeks.

In another embodiment, the maintenance dose is 6 mg/kg of the antibody and this is administered in two divided doses each containing 3 mg/kg of the antibody and the administration interval is two weeks (every 2 weeks). In this case two doses are administered during a month or 4 weeks.

In another embodiment, the maintenance dose is 6 mg/kg of the antibody and this is administered in four divided doses each containing 1.5 mg/kg of the antibody and the administration interval is one week (every week). In this case four doses are administered during a month or 4 weeks.

In certain embodiment, the maintenance dose is 6 mg/kg of the antibody in a single dose and the administration interval is 4 weeks (every month) and in this case one dose is administered during a month or 4 weeks.

In another embodiment, the maintenance dose is 3 mg/kg of the antibody in a single dose and the administration interval is two weeks (every 2 weeks) and in this case two doses are administered during a month or 4 weeks.

In another embodiment, the maintenance dose is 1.5 mg/kg of the antibody in a single dose and the administration interval is one week (every week) and in this case four doses are administered during a month or 4 weeks.

In another aspect, the maintenance dose is 9 mg/kg of the antibody. The maintenance dose of 9 mg/kg refers to, for example, administering total 9 mg/kg of the antibody during 4 weeks or every month in a single dose or multiple divided doses. This can be applicable for pediatric patients or such special population of patients likely to exhibit lower exposure.

In certain embodiment, the maintenance dose is 9 mg/kg of the antibody and this is administered in two divided doses each containing 4.5 mg/kg of the antibody and the administration interval is two weeks (every 2 weeks). In this case two doses are administered during a month or 4 weeks.

In another embodiment, the maintenance dose is 9 mg/kg of the antibody and this is administered in four divided doses each containing 2.25 mg/kg of the antibody and the administration interval is one week (every week). In this case four doses are administered during a month or 4 weeks.

In certain another embodiment, the maintenance dose is 4.5 mg/kg of the antibody in a single dose and the administration interval is two weeks (every 2 weeks) and in this case two doses are administered during a month or 4 weeks.

In another embodiment, the maintenance dose is 2.25 mg/kg of the antibody in a single dose and the administration interval is one week (every week) and in this case four doses are administered during a month or 4 weeks.

In another aspect, the maintenance dose is 12 mg/kg of the antibody. The maintenance dose of 12 mg/kg refers to, for example, administering total 12 mg/kg of the antibody during 4 weeks or every month in a single dose or multiple divided doses. This can be applicable for pediatric patients or such special population of patients likely to exhibit lower exposure.

In certain embodiment, the maintenance dose is 12 mg/kg of the antibody and this is administered in two divided doses each containing 6 mg/kg of the antibody and the administration interval is two weeks (every 2 weeks). In this case two doses are administered during a month or 4 weeks.

In another embodiment, the maintenance dose is 12 mg/kg of the antibody and this is administered in four divided doses each containing 3 mg/kg of the antibody and the administration interval is one week (every week). In this case four doses are administered during a month or 4 weeks.

In certain embodiment, the maintenance dose is 6 mg/kg of the antibody in a single dose and the administration interval is two weeks (every 2 weeks) and in this case two doses are administered during a month or 4 weeks.

In another embodiment, the maintenance dose is 3 mg/kg of the antibody in a single dose and the administration interval is one week (every week) and in this case four doses are administered during a month or 4 weeks.

In another aspect, a different or alternative maintenance dose and administration interval can be applicable.

In certain embodiments, a different or alternative maintenance dose and administration interval can be applicable after the above-mentioned initial maintenance doses and administration intervals. More specifically, one may modify the above-mentioned every-4-week 6 mg/kg antibody maintenance dosing regimen, every-4-week 9 mg/kg antibody maintenance dosing regimen, and/or every-4-week 12 mg/kg antibody maintenance dosing regimen to apply a different, alternative, or modified maintenance dose and administration interval. For example, after carrying out the administration in any of the above-mentioned every-4-week 6 mg/kg antibody maintenance dosing regimen, every-4-week 9 mg/kg antibody maintenance dosing regimen, and every-4-weeks 12 mg/kg antibody maintenance dosing regimen, one may assess whether the administration of the maintenance dose was sufficient to treat the subject. If the administration of the maintenance dose is turned out to be insufficient or to cause no or insufficient treatment effect and/or preventive effect, then, one may stop administering the maintenance dose of the antibody and start administering a modified maintenance dose. The effect of the administration of the maintenance dose may be assessed at least 1, 2, 3, 4, 5, 6, 7, 8, 9, 10, 11, 12, 16, 20, 24, 36, 48, 60, or more weeks after the start of the administration of the maintenance dose. There is no particular limitation on the number of times that one can change the maintenance dose. The changes of the maintenance dose may be made several times, e.g., once to four times. In other words, one to several, e.g. one to five, different maintenance doses may be applied in a sequential manner, such as (0) administering a maintenance dose, (1) stopping administering the maintenance dose and starting administering a first modified maintenance dose, (2) stopping administering the first modified maintenance dose and starting administering a second modified maintenance dose, (3) stopping administering the second modified maintenance dose and starting administering a third modified maintenance dose, (4) stopping administering the third modified maintenance dose and starting administering a fourth modified maintenance dose.

In some embodiments, the modified maintenance dose may be applied from the beginning, without using the above-mentioned every-4-week 6 mg/kg antibody maintenance dosing regimen, every-4-week 9 mg/kg antibody maintenance dosing regimen, and/or every-4-week 12 mg/kg antibody maintenance dosing regimen.

In certain embodiment, the modified maintenance dose is 6 mg/kg of the antibody. The modified maintenance dose of 6 mg/kg refers to, for example, administering total 6 mg/kg of the antibody during 2 weeks or every 2 weeks as a single dose or multiple divided doses.

In certain embodiment, the modified maintenance dose is 6 mg/kg of the antibody and this is administered in a single dose and the modified administration interval is 2 weeks (every 2 weeks). In this case one single dose is administered during 2 weeks.

In another embodiment, the modified maintenance dose is 6 mg/kg of the antibody and this is administered in two divided doses each containing 3 mg/kg of the antibody and the modified administration interval is one week (every week). In this case two doses are administered during 2 weeks.

In certain embodiment, the modified maintenance dose is 6 mg/kg of the antibody in a single dose and the modified administration interval is two weeks (every 2 weeks) and in this case two doses are administered during a month or 4 weeks, or in other words one single dose is administered during 2 weeks.

In another embodiment, the modified maintenance dose is 3 mg/kg of the antibody in a single dose and the modified administration interval is one week (every week) and in this case two doses are administered during 2 weeks.

In certain embodiment, the modified maintenance dose is 9 mg/kg of the antibody. The modified maintenance dose of 9 mg/kg refers to, for example, administering total 9 mg/kg of the antibody during 2 weeks or every 2 weeks as a single dose or multiple divided doses. This can be applicable for pediatric patients or such special population of patients likely to exhibit lower exposure.

In another embodiment, the modified maintenance dose is 9 mg/kg of the antibody and this is administered in two divided doses each containing 4.5 mg/kg of the antibody and the modified administration interval is one week (every week). In this case two doses are administered during 2 weeks.

In another embodiment, the modified maintenance dose is 4.5 mg/kg of the antibody in a single dose and the modified administration interval is one week (every week) and in this case four doses are administered during a month or 4 weeks, or in other words two doses are administered during 2 weeks.

In certain embodiment, the modified maintenance dose is 12 mg/kg of the antibody. The modified maintenance dose of 12 mg/kg refers to, for example, administering total 12 mg/kg of the antibody during 2 weeks or every 2 weeks as a single dose or multiple divided doses. This can be applicable for pediatric patients or such special population of patients likely to exhibit lower exposure.

In another embodiment, the modified maintenance dose is 12 mg/kg of the antibody and this is administered in two divided doses each containing 6 mg/kg of the antibody and the modified administration interval is one week (every week). In this case two doses are administered during 2 weeks.

In another embodiment with referring to each divided dose above as the modified maintenance dose, the modified maintenance dose is 6 mg/kg of the antibody in a single dose and the modified administration interval is one week (every week) and in this case four doses are administered during a month or 4 weeks, or in other words two doses are administered during 2 weeks.

A “loading” dose herein generally comprises an initial dose of a therapeutic agent administered to a patient, and is followed by one or more maintenance dose(s) thereof. In one aspect, the loading dose refers to the amount given at each individual administration and the administration can be carried out between zero time to 24 times, preferably at least once, at least twice, at least three times, at least four times, or more and preferably twice or four times. Usually, the loading doses are administered at spaced treatment intervals, such as between one week to 4 weeks apart and preferably approximately every week, approximately every 2 weeks, approximately every 3 weeks, or approximately every 4 weeks (every month). In one aspect, the loading dose is between 0.3 mg/kg to 30 mg/kg and preferably 3 mg/kg, 4.5 mg/kg, or 6 mg/kg of the antibody. The loading doses were intended to achieve the steady-state therapeutic plasma concentration as early as possible.

In certain embodiment, the loading dose is 3 mg/kg and the administration interval is one week (every week) and the administration is repeated four times.

In another embodiment, the loading dose is 4.5 mg/kg and the administration interval is one week (every week) and the administration is repeated twice or four times.

In another embodiment, the loading dose is 6 mg/kg and the administration interval is one week (every week) and the administration is repeated four times.

In another embodiment, the loading dose is 6 mg/kg and the administration interval is two weeks (every 2 weeks) and the administration is repeated twice.

The number of times the maintenance dose is administered is not particularly limited, and the number is for example at least once, at least twice, at least three times, at least four times, at least five times, at least six times, at least seven times, at least eight times, at least nine times, at least ten times, at least 15 times, at least 20 times, at least 25 times, at least 35 times, at least 40 times, at least 50 times, at least 60 times, at least 70 times, at least 80 times, at least 90 times, at least 100 times, at least 500 times, at least 1000 times, at least 10000 times, or more.

“Administration interval” (an interval between individual administrations) indicates the interval between administration of the n^{th} loading dose (n is an integer of 1 or greater) and administration of the (n+1)^{th} loading dose, and the interval between administration of the n^{th} maintenance dose (n is an integer of 1 or greater) and administration of the (n+1)^{th} maintenance dose.

In certain embodiment, the antibody is administered as follows.
Regimen A: administration at the loading dose of 3 mg/kg of the antibody once every week for four weeks followed by administration at the maintenance dose of 1.5 mg/kg of the antibody once every week (weekly).
Regimen B: administration at the loading dose of 3 mg/kg of the antibody once every week for four weeks followed by administration at the maintenance dose of 1.5 mg/kg of the antibody once every week (weekly) followed by administration at the modified maintenance dose of 3 mg/kg of the antibody once every week.
Regimen C: administration at the loading dose of 3 mg/kg of the antibody once every week for four weeks followed by administration at the maintenance dose of 1.5 mg/kg of the antibody once every week (weekly) followed by administration at the modified maintenance dose of 6 mg/kg of the antibody once every two weeks.
Regimen D: administration at the loading dose of 3 mg/kg of the antibody once every week for four weeks followed by administration at the maintenance dose of 3 mg/kg of the antibody once every two weeks.
Regimen E: administration at the loading dose of 3 mg/kg of the antibody once every week for four weeks followed by administration at the maintenance dose of 3 mg/kg of the antibody once every two weeks followed by administration at the modified maintenance dose of 3 mg/kg of the antibody once every week.
Regimen F: administration at the loading dose of 3 mg/kg of the antibody once every week for four weeks followed by administration at the maintenance dose of 3 mg/kg of the antibody once every two weeks followed by administration at the modified maintenance dose of 6 mg/kg of the antibody once every two weeks.
Regimen G: administration at the loading dose of 3 mg/kg of the antibody once every week for four weeks followed by administration at the maintenance dose of 6 mg/kg of the antibody once every four weeks (every month).
Regimen H: administration at the loading dose of 3 mg/kg of the antibody once every week for four weeks followed by administration at the maintenance dose of 6 mg/kg of the antibody once every four weeks (every month) followed by administration at the modified maintenance dose of 3 mg/kg of the antibody once every week.
Regimen I: administration at the loading dose of 3 mg/kg of the antibody once every week for four weeks followed by administration at the maintenance dose of 6 mg/kg of the antibody once every four weeks (every month) followed by administration at the modified maintenance dose of 6 mg/kg of the antibody once every two weeks.
Regimen J: administration at the loading dose of 4.5 mg/kg of the antibody once every week for two weeks followed by administration at the maintenance dose of 1.5 mg/kg of the antibody once every week (weekly).
Regimen K: administration at the loading dose of 4.5 mg/kg of the antibody once every week for two weeks followed by administration at the maintenance dose of 1.5 mg/kg of the antibody once every week (weekly) followed by administration at the modified maintenance dose of 3 mg/kg of the antibody once every week.
Regimen L: administration at the loading dose of 4.5 mg/kg of the antibody once every week for two weeks followed by administration at the maintenance dose of 1.5 mg/kg of the antibody once every week (weekly) followed by administration at the modified maintenance dose of 6 mg/kg of the antibody once every two weeks.

1Regimen M: administration at the loading dose of 4.5 mg/kg of the antibody once every week for two weeks followed by administration at the maintenance dose of 3 mg/kg of the antibody once every two weeks.
Regimen N: administration at the loading dose of 4.5 mg/kg of the antibody once every week for two weeks followed by administration at the maintenance dose of 3 mg/kg of the antibody once every two weeks followed by administration at the modified maintenance dose of 3 mg/kg of the antibody once every week.
Regimen O: administration at the loading dose of 4.5 mg/kg of the antibody once every week for two weeks followed by administration at the maintenance dose of 3 mg/kg of the antibody once every two weeks followed by administration at the modified maintenance dose of 6 mg/kg of the antibody once every two weeks.
Regimen P: administration at the loading dose of 4.5 mg/kg of the antibody once every week for two weeks followed by administration at the maintenance dose of 6 mg/kg of the antibody once every four weeks (every month).
Regimen Q: administration at the loading dose of 4.5 mg/kg of the antibody once every week for two weeks followed by administration at the maintenance dose of 6 mg/kg of the antibody once every four weeks (every month) followed by administration at the modified maintenance dose of 3 mg/kg of the antibody once every week.
Regimen R: administration at the loading dose of 4.5 mg/kg of the antibody once every week for two weeks followed by administration at the maintenance dose of 6 mg/kg of the antibody once every four weeks (every month) followed by administration at the modified maintenance dose of 6 mg/kg of the antibody once every two weeks.
Regimen S: administration at the loading dose of 6 mg/kg of the antibody once every two weeks for four weeks followed by administration at the maintenance dose of 1.5 mg/kg of the antibody once every week (weekly).
Regimen T: administration at the loading dose of 6 mg/kg of the antibody once every two weeks for four weeks followed by administration at the maintenance dose of 1.5 mg/kg of the antibody once every week (weekly) followed by administration at the modified maintenance dose of 3 mg/kg of the antibody once every week.
Regimen U: administration at the loading dose of 6 mg/kg of the antibody once every two weeks for four weeks followed by administration at the maintenance dose of 1.5 mg/kg of the antibody once every week (weekly) followed by administration at the modified maintenance dose of 6 mg/kg of the antibody once every two weeks.
Regimen V: administration at the loading dose of 6 mg/kg of the antibody once every two weeks for four weeks followed by administration at the maintenance dose of 3 mg/kg of the antibody once every two weeks.
Regimen W: administration at the loading dose of 6 mg/kg of the antibody once every two weeks for four weeks followed by administration at the maintenance dose of 3 mg/kg of the antibody once every two weeks followed by administration at the modified maintenance dose of 3 mg/kg of the antibody once every week.
Regimen X: administration at the loading dose of 6 mg/kg of the antibody once every two weeks for four weeks followed by administration at the maintenance dose of 3 mg/kg of the antibody once every two weeks followed by administration at the modified maintenance dose of 6 mg/kg of the antibody once every two weeks.
Regimen Y: administration at the loading dose of 6 mg/kg of the antibody once every two weeks for four weeks followed by administration at the maintenance dose of 6 mg/kg of the antibody once every four weeks (every month).
Regimen Z: administration at the loading dose of 6 mg/kg of the antibody once every two weeks for four weeks followed by administration at the maintenance dose of 6 mg/kg of the antibody once every four weeks (every month) followed by administration at the modified maintenance dose of 3 mg/kg of the antibody once every week.
Regimen AA: administration at the loading dose of 6 mg/kg of the antibody once every two weeks for four weeks followed by administration at the maintenance dose of 6 mg/kg of the antibody once every four weeks (every month) followed by administration at the modified maintenance dose of 6 mg/kg of the antibody once every two weeks.
Regimen AB: administration at the loading dose of 4.5 mg/kg of the antibody once every week (weekly) for four weeks followed by administration at the maintenance dose of 2.25 mg/kg of the antibody once every week (weekly).
Regimen AC: administration at the loading dose of 4.5 mg/kg of the antibody once every week (weekly) for four weeks followed by administration at the maintenance dose of 2.25 mg/kg of the antibody once every week (weekly) followed by administration at the modified maintenance dose of 4.5 mg/kg of the antibody once every week (weekly).
Regimen AD: administration at the loading dose of 4.5 mg/kg of the antibody once every week (weekly) for four weeks followed by administration at the maintenance dose of 4.5 mg/kg of the antibody once every two weeks.
Regimen AE: administration at the loading dose of 4.5 mg/kg of the antibody once every week (weekly) for four weeks followed by administration at the maintenance dose of 4.5 mg/kg of the antibody once every two weeks followed by administration at the modified maintenance dose of 4.5 mg/kg of the antibody once every week (weekly).
Regimen AF: administration at the loading dose of 6 mg/kg of the antibody once every week (weekly) for four weeks followed by administration at the maintenance dose of 3 mg/kg of the antibody once every week (weekly).
Regimen AG: administration at the loading dose of 6 mg/kg of the antibody once every week (weekly) for four weeks followed by administration at the maintenance dose of 3 mg/kg of the antibody once every week (weekly) followed by administration at the modified maintenance dose of 6 mg/kg of the antibody once every week (weekly).
Regimen AH: administration at the loading dose of 6 mg/kg of the antibody once every week (weekly) for four weeks followed by administration at the maintenance dose of 6 mg/kg of the antibody once every two weeks.
Regimen AI: administration at the loading dose of 6 mg/kg of the antibody once every week (weekly) for four weeks followed by administration at the maintenance dose of 6 mg/kg of the antibody once every two weeks followed by administration at the modified maintenance dose of 6 mg/kg of the antibody once every week (weekly).
Regimen AJ: administration at the loading dose of 4.5 mg/kg of the antibody once every week (weekly) for four weeks followed by administration at the maintenance dose of 2.25 mg/kg of the antibody once every week (weekly) followed by administration at the modified maintenance dose of 9 mg/kg of the antibody once every two weeks.
Regimen AK: administration at the loading dose of 4.5 mg/kg of the antibody once every week (weekly) for four weeks followed by administration at the maintenance dose of 4.5 mg/kg of the antibody once every two weeks followed by administration at the modified maintenance dose of 9 mg/kg of the antibody once every two weeks.
Regimen AL: administration at the loading dose of 4.5 mg/kg of the antibody once every week (weekly) for four weeks followed by administration at the maintenance dose of 9 mg/kg of the antibody once every four weeks (every month).
Regimen AM: administration at the loading dose of 4.5 mg/kg of the antibody once every week (weekly) for four weeks followed by administration at the maintenance dose of 9 mg/kg of the antibody once every four weeks (every month) followed by administration at the modified maintenance dose of 4.5 mg/kg of the antibody once every week (weekly).
Regimen AN: administration at the loading dose of 4.5 mg/kg of the antibody once every week (weekly) for four weeks followed by administration at the maintenance dose of 9 mg/kg of the antibody once every four weeks (every month) followed by administration at the modified maintenance dose of 9 mg/kg of the antibody once every two weeks.
Regimen AO: administration at the loading dose of 6.75 mg/kg of the antibody once every week (weekly) for two weeks followed by administration at the maintenance dose of 2.25 mg/kg of the antibody once every week (weekly).
Regimen AP: administration at the loading dose of 6.75 mg/kg of the antibody once every week (weekly) for two weeks followed by administration at the maintenance dose of 2.25 mg/kg of the antibody once every week (weekly) followed by administration at the modified maintenance dose of 4.5 mg/kg of the antibody once every week (weekly).
Regimen AQ: administration at the loading dose of 6.75 mg/kg of the antibody once every week (weekly) for two weeks followed by administration at the maintenance dose of 2.25 mg/kg of the antibody once every week (weekly) followed by administration at the modified maintenance dose of 9 mg/kg of the antibody once every two weeks.
Regimen AR: administration at the loading dose of 6.75 mg/kg of the antibody once every week (weekly) for two weeks followed by administration at the maintenance dose of 4.5 mg/kg of the antibody once every two weeks.
Regimen AS: administration at the loading dose of 6.75 mg/kg of the antibody once every week (weekly) for two weeks followed by administration at the maintenance dose of 4.5 mg/kg of the antibody once every two weeks followed by administration at the modified maintenance dose of 4.5 mg/kg of the antibody once every week (weekly).
Regimen AT: administration at the loading dose of 6.75 mg/kg of the antibody once every week (weekly) for two weeks followed by administration at the maintenance dose of 4.5 mg/kg of the antibody once every two weeks followed by administration at the modified maintenance dose of 9 mg/kg of the antibody once every two weeks.
Regimen AU: administration at the loading dose of 6.75 mg/kg of the antibody once every week (weekly) for two weeks followed by administration at the maintenance dose of 9 mg/kg of the antibody once every four weeks (every month).
Regimen AV: administration at the loading dose of 6.75 mg/kg of the antibody once every week (weekly) for two weeks followed by administration at the maintenance dose of 9 mg/kg of the antibody once every four weeks (every month) followed by administration at the modified maintenance dose of 4.5 mg/kg of the antibody once every week (weekly).
Regimen AW: administration at the loading dose of 6.75 mg/kg of the antibody once every week (weekly) for two weeks followed by administration at the maintenance dose of 9 mg/kg of the antibody once every four weeks (every month) followed by administration at the modified maintenance dose of 9 mg/kg of the antibody once every two weeks.
Regimen AX: administration at the loading dose of 9 mg/kg of the antibody once every two weeks for four weeks followed by administration at the maintenance dose of 2.25 mg/kg of the antibody once every week (weekly).
Regimen AY: administration at the loading dose of 9 mg/kg of the antibody once every two weeks for four weeks followed by administration at the maintenance dose of 2.25 mg/kg of the antibody once every week (weekly) followed by administration at the modified maintenance dose of 4.5 mg/kg of the antibody once every week (weekly).
Regimen AZ: administration at the loading dose of 9 mg/kg of the antibody once every two weeks for four weeks followed by administration at the maintenance dose of 2.25 mg/kg of the antibody once every week (weekly) followed by administration at the modified maintenance dose of 9 mg/kg of the antibody once every two weeks.
Regimen BA: administration at the loading dose of 9 mg/kg of the antibody once every two weeks for four weeks followed by administration at the maintenance dose of 4.5 mg/kg of the antibody once every two weeks.
Regimen BB: administration at the loading dose of 9 mg/kg of the antibody once every two weeks for four weeks followed by administration at the maintenance dose of 4.5 mg/kg of the antibody once every two weeks followed by administration at the modified maintenance dose of 4.5 mg/kg of the antibody once every week (weekly).
Regimen BC: administration at the loading dose of 9 mg/kg of the antibody once every two weeks for four weeks followed by administration at the maintenance dose of 4.5 mg/kg of the antibody once every two weeks followed by administration at the modified maintenance dose of 9 mg/kg of the antibody once every two weeks.
Regimen BD: administration at the loading dose of 9 mg/kg of the antibody once every two weeks for four weeks followed by administration at the maintenance dose of 9 mg/kg of the antibody once every four weeks (every month).
Regimen BE: administration at the loading dose of 9 mg/kg of the antibody once every two weeks for four weeks followed by administration at the maintenance dose of 9 mg/kg of the antibody once every four weeks (every month) followed by administration at the modified maintenance dose of 4.5 mg/kg of the antibody once every week (weekly).
Regimen BF: administration at the loading dose of 9 mg/kg of the antibody once every two weeks for four weeks followed by administration at the maintenance dose of 9 mg/kg of the antibody once every four weeks (every month) followed by administration at the modified maintenance dose of 9 mg/kg of the antibody once every two weeks.
Regimen BG: administration at the loading dose of 6 mg/kg of the antibody once every week (weekly) for four weeks followed by administration at the maintenance dose of 3 mg/kg of the antibody once every week (weekly) followed by administration at the modified maintenance dose of 12 mg/kg of the antibody once every two weeks.
Regimen BH: administration at the loading dose of 6 mg/kg of the antibody once every week (weekly) for four weeks followed by administration at the maintenance dose of 6 mg/kg of the antibody once every two weeks followed by administration at the modified maintenance dose of 12 mg/kg of the antibody once every two weeks.
Regimen BI: administration at the loading dose of 6 mg/kg of the antibody once every week (weekly) for four weeks followed by administration at the maintenance dose of 12 mg/kg of the antibody once every four weeks (every month).
Regimen BJ: administration at the loading dose of 6 mg/kg of the antibody once every week (weekly) for four weeks followed by administration at the maintenance dose of 12 mg/kg of the antibody once every four weeks (every month) followed by administration at the modified maintenance dose of 6 mg/kg of the antibody once every week (weekly).
Regimen BK: administration at the loading dose of 6 mg/kg of the antibody once every week (weekly) for four weeks followed by administration at the maintenance dose of 12 mg/kg of the antibody once every four weeks (every month) followed by administration at the modified maintenance dose of 12 mg/kg of the antibody once every two weeks.
Regimen BL: administration at the loading dose of 9 mg/kg of the antibody once every week (weekly) for two weeks followed by administration at the maintenance dose of 3 mg/kg of the antibody once every week (weekly).
Regimen BM: administration at the loading dose of 9 mg/kg of the antibody once every week (weekly) for two weeks followed by administration at the maintenance dose of 3 mg/kg of the antibody once every week (weekly) followed by administration at the modified maintenance dose of 6 mg/kg of the antibody once every week (weekly).
Regimen BN: administration at the loading dose of 9 mg/kg of the antibody once every week (weekly) for two weeks followed by administration at the maintenance dose of 3 mg/kg of the antibody once every week (weekly) followed by administration at the modified maintenance dose of 12 mg/kg of the antibody once every two weeks.
Regimen BO: administration at the loading dose of 9 mg/kg of the antibody once every week (weekly) for two weeks followed by administration at the maintenance dose of 6 mg/kg of the antibody once every two weeks.
Regimen BP: administration at the loading dose of 9 mg/kg of the antibody once every week (weekly) for two weeks followed by administration at the maintenance dose of 6 mg/kg of the antibody once every two weeks followed by administration at the modified maintenance dose of 6 mg/kg of the antibody once every week (weekly).
Regimen BQ: administration at the loading dose of 9 mg/kg of the antibody once every week (weekly) for two weeks followed by administration at the maintenance dose of 6 mg/kg of the antibody once every two weeks followed by administration at the modified maintenance dose of 12 mg/kg of the antibody once every two weeks.
Regimen BR: administration at the loading dose of 9 mg/kg of the antibody once every week (weekly) for two weeks followed by administration at the maintenance dose of 12 mg/kg of the antibody once every four weeks (every month).
Regimen BS: administration at the loading dose of 9 mg/kg of the antibody once every week (weekly) for two weeks followed by administration at the maintenance dose of 12 mg/kg of the antibody once every four weeks (every month) followed by administration at the modified maintenance dose of 6 mg/kg of the antibody once every week (weekly).
Regimen BT: administration at the loading dose of 9 mg/kg of the antibody once every week (weekly) for two weeks followed by administration at the maintenance dose of 12 mg/kg of the antibody once every four weeks (every month) followed by administration at the modified maintenance dose of 12 mg/kg of the antibody once every two weeks.
Regimen BU: administration at the loading dose of 12 mg/kg of the antibody once every two weeks for four weeks followed by administration at the maintenance dose of 3 mg/kg of the antibody once every week (weekly).
Regimen BV: administration at the loading dose of 12 mg/kg of the antibody once every two weeks for four weeks followed by administration at the maintenance dose of 3 mg/kg of the antibody once every week (weekly) followed by administration at the modified maintenance dose of 6 mg/kg of the antibody once every week (weekly).
Regimen BW: administration at the loading dose of 12 mg/kg of the antibody once every two weeks for four weeks followed by administration at the maintenance dose of 3 mg/kg of the antibody once every week (weekly) followed by administration at the modified maintenance dose of 12 mg/kg of the antibody once every two weeks.
Regimen BX: administration at the loading dose of 12 mg/kg of the antibody once every two weeks for four weeks followed by administration at the maintenance dose of 6 mg/kg of the antibody once every two weeks.
Regimen BY: administration at the loading dose of 12 mg/kg of the antibody once every two weeks for four weeks followed by administration at the maintenance dose of 6 mg/kg of the antibody once every two weeks followed by administration at the modified maintenance dose of 6 mg/kg of the antibody once every week (weekly).
Regimen BZ: administration at the loading dose of 12 mg/kg of the antibody once every two weeks for four weeks followed by administration at the maintenance dose of 6 mg/kg of the antibody once every two weeks followed by administration at the modified maintenance dose of 12 mg/kg of the antibody once every two weeks.
Regimen CA: administration at the loading dose of 12 mg/kg of the antibody once every two weeks for four weeks followed by administration at the maintenance dose of 12 mg/kg of the antibody once every four weeks (every month).
Regimen CB: administration at the loading dose of 12 mg/kg of the antibody once every two weeks for four weeks followed by administration at the maintenance dose of 12 mg/kg of the antibody once every four weeks (every month) followed by administration at the modified maintenance dose of 6 mg/kg of the antibody once every week (weekly).
Regimen CC: administration at the loading dose of 12 mg/kg of the antibody once every two weeks for four weeks followed by administration at the maintenance dose of 12 mg/kg of the antibody once every four weeks (every month) followed by administration at the modified maintenance dose of 12 mg/kg of the antibody once every two weeks.
Regimen CD: administration at the maintenance dose of 3 mg/kg of the antibody once every week (weekly).
Regimen CE: administration at the maintenance dose of 3 mg/kg of the antibody once every week (weekly) followed by administration at the modified maintenance dose of 4.5 mg/kg of the antibody once every week (weekly).
Regimen CF: administration at the maintenance dose of 3 mg/kg of the antibody once every week (weekly) followed by administration at the modified maintenance dose of 9 mg/kg of the antibody once every two weeks.
Regimen CG: administration at the maintenance dose of 3 mg/kg of the antibody once every week (weekly) followed by administration at the first modified maintenance dose of 4.5 mg/kg of the antibody once every week (weekly) followed by administration at the second modified maintenance dose of 6 mg/kg of the antibody once every week (weekly).
Regimen CH: administration at the maintenance dose of 3 mg/kg of the antibody once every week (weekly) followed by administration at the first modified maintenance dose of 4.5 mg/kg of the antibody once every week (weekly) followed by administration at the second modified maintenance dose of 12 mg/kg of the antibody once every two weeks.
Regimen CI: administration at the maintenance dose of 3 mg/kg of the antibody once every week (weekly) followed by administration at the first modified maintenance dose of 9 mg/kg of the antibody once every two weeks followed by administration at the second modified maintenance dose of 6 mg/kg of the antibody once every week (weekly).
Regimen CJ: administration at the maintenance dose of 3 mg/kg of the antibody once every week (weekly) followed by administration at the first modified maintenance dose of 9 mg/kg of the antibody once every two weeks followed by administration at the second modified maintenance dose of 12 mg/kg of the antibody once every two weeks.
Regimen CK: administration at the maintenance dose of 3 mg/kg of the antibody once every week (weekly) followed by administration at the modified maintenance dose of 6 mg/kg of the antibody once every week (weekly).
Regimen CL: administration at the maintenance dose of 3 mg/kg of the antibody once every week (weekly) followed by administration at the modified maintenance dose of 12 mg/kg of the antibody once every two weeks.
Regimen CM: administration at the maintenance dose of 6 mg/kg of the antibody once every two weeks.
Regimen CN: administration at the maintenance dose of 6 mg/kg of the antibody once every two weeks followed by administration at the modified maintenance dose of 4.5 mg/kg of the antibody once every week (weekly).
Regimen CO: administration at the maintenance dose of 6 mg/kg of the antibody once every two weeks followed by administration at the modified maintenance dose of 9 mg/kg of the antibody once every two weeks.
Regimen CP: administration at the maintenance dose of 6 mg/kg of the antibody once every two weeks followed by administration at the first modified maintenance dose of 4.5 mg/kg of the antibody once every week (weekly) followed by administration at the second modified maintenance dose of 6 mg/kg of the antibody once every week (weekly).
Regimen CQ: administration at the maintenance dose of 6 mg/kg of the antibody once every two weeks followed by administration at the first modified maintenance dose of 4.5 mg/kg of the antibody once every week (weekly) followed by administration at the second modified maintenance dose of 12 mg/kg of the antibody once every two weeks.
Regimen CR: administration at the maintenance dose of 6 mg/kg of the antibody once every two weeks followed by administration at the first modified maintenance dose of 9 mg/kg of the antibody once every two weeks followed by administration at the second modified maintenance dose of 6 mg/kg of the antibody once every week (weekly).
Regimen CS: administration at the maintenance dose of 6 mg/kg of the antibody once every two weeks followed by administration at the first modified maintenance dose of 9 mg/kg of the antibody once every two weeks followed by administration at the second modified maintenance dose of 12 mg/kg of the antibody once every two weeks.
Regimen CT: administration at the maintenance dose of 6 mg/kg of the antibody once every two weeks followed by administration at the modified maintenance dose of 6 mg/kg of the antibody once every week (weekly).
Regimen CU: administration at the maintenance dose of 6 mg/kg of the antibody once every two weeks followed by administration at the modified maintenance dose of 12 mg/kg of the antibody once every two weeks.
Regimen CV: administration at the maintenance dose of 12 mg/kg of the antibody once every four weeks (every month).
Regimen CW: administration at the maintenance dose of 12 mg/kg of the antibody once every four weeks (every month) followed by administration at the modified maintenance dose of 4.5 mg/kg of the antibody once every week (weekly).
Regimen CX: administration at the maintenance dose of 12 mg/kg of the antibody once every four weeks (every month) followed by administration at the modified maintenance dose of 9 mg/kg of the antibody once every two weeks.
Regimen CY: administration at the maintenance dose of 12 mg/kg of the antibody once every four weeks (every month) followed by administration at the first modified maintenance dose of 4.5 mg/kg of the antibody once every week (weekly) followed by administration at the second modified maintenance dose of 6 mg/kg of the antibody once every week (weekly).
Regimen CZ: administration at the maintenance dose of 12 mg/kg of the antibody once every four weeks (every month) followed by administration at the first modified maintenance dose of 4.5 mg/kg of the antibody once every week (weekly) followed by administration at the second modified maintenance dose of 12 mg/kg of the antibody once every two weeks.
Regimen DA: administration at the maintenance dose of 12 mg/kg of the antibody once every four weeks (every month) followed by administration at the first modified maintenance dose of 9 mg/kg of the antibody once every two weeks followed by administration at the second modified maintenance dose of 6 mg/kg of the antibody once every week (weekly).
Regimen DB: administration at the maintenance dose of 12 mg/kg of the antibody once every four weeks (every month) followed by administration at the first modified maintenance dose of 9 mg/kg of the antibody once every two weeks followed by administration at the second modified maintenance dose of 12 mg/kg of the antibody once every two weeks.
Regimen DC: administration at the maintenance dose of 12 mg/kg of the antibody once every four weeks (every month) followed by administration at the modified maintenance dose of 6 mg/kg of the antibody once every week (weekly).
Regimen DD: administration at the maintenance dose of 12 mg/kg of the antibody once every four weeks (every month) followed by administration at the modified maintenance dose of 12 mg/kg of the antibody once every two weeks.
Regimen DE: administration at the maintenance dose of 4.5 mg/kg of the antibody once every week (weekly).
Regimen DF: administration at the maintenance dose of 4.5 mg/kg of the antibody once every week (weekly) followed by administration at the modified maintenance dose of 6 mg/kg of the antibody once every week (weekly).
Regimen DG: administration at the maintenance dose of 4.5 mg/kg of the antibody once every week (weekly) followed by administration at the modified maintenance dose of 12 mg/kg of the antibody once every two weeks.
Regimen DH: administration at the maintenance dose of 9 mg/kg of the antibody once every two weeks.
Regimen DI: administration at the maintenance dose of 9 mg/kg of the antibody once every two weeks followed by administration at the modified maintenance dose of 6 mg/kg of the antibody once every week (weekly).
Regimen DJ: administration at the maintenance dose of 9 mg/kg of the antibody once every two weeks followed by administration at the modified maintenance dose of 12 mg/kg of the antibody once every two weeks.
Regimen DK: administration at the maintenance dose of 6 mg/kg of the antibody once every week (weekly).
Regimen DL: administration at the maintenance dose of 12 mg/kg of the antibody once every two weeks.
Regimen DM: administration at the loading dose of 3 mg/kg of the antibody once every week (weekly) for four weeks followed by administration at the maintenance dose of 1.5 mg/kg of the antibody once every week (weekly) followed by administration at the modified maintenance dose of 2.25 mg/kg of the antibody once every week (weekly).
Regimen DN: administration at the loading dose of 3 mg/kg of the antibody once every week (weekly) for four weeks followed by administration at the maintenance dose of 1.5 mg/kg of the antibody once every week (weekly) followed by administration at the first modified maintenance dose of 2.25 mg/kg of the antibody once every week (weekly) followed by administration at the second modified maintenance dose of 3 mg/kg of the antibody once every week (weekly).
Regimen DO: administration at the loading dose of 3 mg/kg of the antibody once every week (weekly) for four weeks followed by administration at the maintenance dose of 1.5 mg/kg of the antibody once every week (weekly) followed by administration at the first modified maintenance dose of 2.25 mg/kg of the antibody once every week (weekly) followed by administration at the second modified maintenance dose of 3 mg/kg of the antibody once every week (weekly) followed by administration at the third modified maintenance dose of 4.5 mg/kg of the antibody once every week (weekly).
Regimen DP: administration at the loading dose of 3 mg/kg of the antibody once every week (weekly) for four weeks followed by administration at the maintenance dose of 1.5 mg/kg of the antibody once every week (weekly) followed by administration at the first modified maintenance dose of 2.25 mg/kg of the antibody once every week (weekly) followed by administration at the second modified maintenance dose of 3 mg/kg of the antibody once every week (weekly) followed by administration at the third modified maintenance dose of 4.5 mg/kg of the antibody once every week (weekly) followed by administration at the fourth modified maintenance dose of 6 mg/kg of the antibody once every week (weekly).
Regimen DQ: administration at the loading dose of 3 mg/kg of the antibody once every week (weekly) for four weeks followed by administration at the maintenance dose of 1.5 mg/kg of the antibody once every week (weekly) followed by administration at the first modified maintenance dose of 2.25 mg/kg of the antibody once every week (weekly) followed by administration at the second modified maintenance dose of 3 mg/kg of the antibody once every week (weekly) followed by administration at the third modified maintenance dose of 6 mg/kg of the antibody once every week (weekly).
Regimen DR: administration at the loading dose of 3 mg/kg of the antibody once every week (weekly) for four weeks followed by administration at the maintenance dose of 1.5 mg/kg of the antibody once every week (weekly) followed by administration at the first modified maintenance dose of 2.25 mg/kg of the antibody once every week (weekly) followed by administration at the second modified maintenance dose of 4.5 mg/kg of the antibody once every week (weekly).
Regimen DS: administration at the loading dose of 3 mg/kg of the antibody once every week (weekly) for four weeks followed by administration at the maintenance dose of 1.5 mg/kg of the antibody once every week (weekly) followed by administration at the first modified maintenance dose of 2.25 mg/kg of the antibody once every week (weekly) followed by administration at the second modified maintenance dose of 4.5 mg/kg of the antibody once every week (weekly) followed by administration at the third modified maintenance dose of 6 mg/kg of the antibody once every week (weekly).
Regimen DT: administration at the loading dose of 3 mg/kg of the antibody once every week (weekly) for four weeks followed by administration at the maintenance dose of 1.5 mg/kg of the antibody once every week (weekly) followed by administration at the first modified maintenance dose of 3 mg/kg of the antibody once every week (weekly) followed by administration at the second modified maintenance dose of 4.5 mg/kg of the antibody once every week (weekly).
Regimen DU: administration at the loading dose of 3 mg/kg of the antibody once every week (weekly) for four weeks followed by administration at the maintenance dose of 1.5 mg/kg of the antibody once every week (weekly) followed by administration at the first modified maintenance dose of 3 mg/kg of the antibody once every week (weekly) followed by administration at the second modified maintenance dose of 4.5 mg/kg of the antibody once every week (weekly) followed by administration at the third modified maintenance dose of 6 mg/kg of the antibody once every week (weekly).
Regimen DV: administration at the loading dose of 3 mg/kg of the antibody once every week (weekly) for four weeks followed by administration at the maintenance dose of 1.5 mg/kg of the antibody once every week (weekly) followed by administration at the first modified maintenance dose of 3 mg/kg of the antibody once every week (weekly) followed by administration at the second modified maintenance dose of 6 mg/kg of the antibody once every week (weekly).
Regimen DW: administration at the loading dose of 3 mg/kg of the antibody once every week (weekly) for four weeks followed by administration at the maintenance dose of 2.25 mg/kg of the antibody once every week (weekly).
Regimen DX: administration at the loading dose of 3 mg/kg of the antibody once every week (weekly) for four weeks followed by administration at the maintenance dose of 2.25 mg/kg of the antibody once every week (weekly) followed by administration at the modified maintenance dose of 3 mg/kg of the antibody once every week (weekly).
Regimen DY: administration at the loading dose of 3 mg/kg of the antibody once every week (weekly) for four weeks followed by administration at the maintenance dose of 2.25 mg/kg of the antibody once every week (weekly) followed by administration at the first modified maintenance dose of 3 mg/kg of the antibody once every week (weekly) followed by administration at the second modified maintenance dose of 4.5 mg/kg of the antibody once every week (weekly).
Regimen DZ: administration at the loading dose of 3 mg/kg of the antibody once every week (weekly) for four weeks followed by administration at the maintenance dose of 2.25 mg/kg of the antibody once every week (weekly) followed by administration at the first modified maintenance dose of 3 mg/kg of the antibody once every week (weekly) followed by administration at the second modified maintenance dose of 4.5 mg/kg of the antibody once every week (weekly) followed by administration at the third modified maintenance dose of 6 mg/kg of the antibody once every week (weekly).
Regimen EA: administration at the loading dose of 3 mg/kg of the antibody once every week (weekly) for four weeks followed by administration at the maintenance dose of 2.25 mg/kg of the antibody once every week (weekly) followed by administration at the first modified maintenance dose of 3 mg/kg of the antibody once every week (weekly) followed by administration at the second modified maintenance dose of 6 mg/kg of the antibody once every week (weekly).
Regimen EB: administration at the loading dose of 3 mg/kg of the antibody once every week (weekly) for four weeks followed by administration at the maintenance dose of 2.25 mg/kg of the antibody once every week (weekly) followed by administration at the modified maintenance dose of 4.5 mg/kg of the antibody once every week (weekly).
Regimen EC: administration at the loading dose of 3 mg/kg of the antibody once every week (weekly) for four weeks followed by administration at the maintenance dose of 2.25 mg/kg of the antibody once every week (weekly) followed by administration at the first modified maintenance dose of 4.5 mg/kg of the antibody once every week (weekly) followed by administration at the second modified maintenance dose of 6 mg/kg of the antibody once every week (weekly).
Regimen ED: (I) administration at the loading dose of (I-i) 3 mg/kg of the antibody once every week (weekly) for four weeks, (I-ii) 4.5 mg/kg of the antibody once every week (weekly) for two weeks, or (I-iii) 6 mg/kg of the antibody once every two weeks for four weeks, followed by (II) administration at the maintenance dose of (II-i) 1.5 mg/kg of the antibody once every week (weekly), (II-ii) 3 mg/kg of the antibody once every two weeks, or (II-iii) 6 mg/kg of the antibody once every four weeks (every month), followed by (III) administration at the modified maintenance dose of (III-i) 2.25 mg/kg of the antibody once every week (weekly), (III-ii) 4.5 mg/kg of the antibody once every two weeks, or (III-iii) 9 mg/kg of the antibody once every four weeks (every month).
Regimen EE: (I) administration at the loading dose of (I-i) 3 mg/kg of the antibody once every week (weekly) for four weeks, (I-ii) 4.5 mg/kg of the antibody once every week (weekly) for two weeks, or (I-iii) 6 mg/kg of the antibody once every two weeks for four weeks, followed by (II) administration at the maintenance dose of (II-i) 1.5 mg/kg of the antibody once every week (weekly), (II-ii) 3 mg/kg of the antibody once every two weeks, or (II-iii) 6 mg/kg of the antibody once every four weeks (every month), followed by (III) administration at the first modified maintenance dose of (III-i) 2.25 mg/kg of the antibody once every week (weekly), (III-ii) 4.5 mg/kg of the antibody once every two weeks, or (III-iii) 9 mg/kg of the antibody once every four weeks (every month), followed by (IV) administration at the second modified maintenance dose of (IV-i) 3 mg/kg of the antibody once every week (weekly), (IV-ii) 6 mg/kg of the antibody once every two weeks, or (IV-iii) 12 mg/kg of the antibody once every four weeks (every month).
Regimen EF: (I) administration at the loading dose of (I-i) 3 mg/kg of the antibody once every week (weekly) for four weeks, (I-ii) 4.5 mg/kg of the antibody once every week (weekly) for two weeks, or (I-iii) 6 mg/kg of the antibody once every two weeks for four weeks, followed by (II) administration at the maintenance dose of (II-i) 1.5 mg/kg of the antibody once every week (weekly), (II-ii) 3 mg/kg of the antibody once every two weeks, or (II-iii) 6 mg/kg of the antibody once every four weeks (every month), followed by (III) administration at the first modified maintenance dose of (III-i) 2.25 mg/kg of the antibody once every week (weekly), (III-ii) 4.5 mg/kg of the antibody once every two weeks, or (III-iii) 9 mg/kg of the antibody once every four weeks (every month), followed by (IV) administration at the second modified maintenance dose of (IV-i) 3 mg/kg of the antibody once every week (weekly), (IV-ii) 6 mg/kg of the antibody once every two weeks, or (IV-iii) 12 mg/kg of the antibody once every four weeks (every month), followed by (V) administration at the third modified maintenance dose of (V-i) 4.5 mg/kg of the antibody once every week (weekly), or (V-ii) 9 mg/kg of the antibody once every two weeks.
Regimen EG: (I) administration at the loading dose of (I-i) 3 mg/kg of the antibody once every week (weekly) for four weeks, (I-ii) 4.5 mg/kg of the antibody once every week (weekly) for two weeks, or (I-iii) 6 mg/kg of the antibody once every two weeks for four weeks, followed by (II) administration at the maintenance dose of (II-i) 1.5 mg/kg of the antibody once every week (weekly), (II-ii) 3 mg/kg of the antibody once every two weeks, or (II-iii) 6 mg/kg of the antibody once every four weeks (every month), followed by (III) administration at the first modified maintenance dose of (III-i) 2.25 mg/kg of the antibody once every week (weekly), (III-ii) 4.5 mg/kg of the antibody once every two weeks, or (III-iii) 9 mg/kg of the antibody once every four weeks (every month), followed by (IV) administration at the second modified maintenance dose of (IV-i) 3 mg/kg of the antibody once every week (weekly), (IV-ii) 6 mg/kg of the antibody once every two weeks, or (IV-iii) 12 mg/kg of the antibody once every four weeks (every month), followed by (V) administration at the third modified maintenance dose of (V-i) 4.5 mg/kg of the antibody once every week (weekly), or (V-ii) 9 mg/kg of the antibody once every two weeks, followed by (VI) administration at the fourth modified maintenance dose of (VI-i) 6 mg/kg of the antibody once every week (weekly), or (VI-ii) 12 mg/kg of the antibody once every two weeks.
Regimen EH: (I) administration at the loading dose of (I-i) 3 mg/kg of the antibody once every week (weekly) for four weeks, (I-ii) 4.5 mg/kg of the antibody once every week (weekly) for two weeks, or (I-iii) 6 mg/kg of the antibody once every two weeks for four weeks, followed by (II) administration at the maintenance dose of (II-i) 1.5 mg/kg of the antibody once every week (weekly), (II-ii) 3 mg/kg of the antibody once every two weeks, or (II-iii) 6 mg/kg of the antibody once every four weeks (every month), followed by (III) administration at the first modified maintenance dose of (III-i) 2.25 mg/kg of the antibody once every week (weekly), (III-ii) 4.5 mg/kg of the antibody once every two weeks, or (III-iii) 9 mg/kg of the antibody once every four weeks (every month), followed by (IV) administration at the second modified maintenance dose of (IV-i) 3 mg/kg of the antibody once every week (weekly), (IV-ii) 6 mg/kg of the antibody once every two weeks, or (IV-iii) 12 mg/kg of the antibody once every four weeks (every month), followed by (V) administration at the third modified maintenance dose of (V-i) 6 mg/kg of the antibody once every week (weekly), or (V-ii) 12 mg/kg of the antibody once every two weeks.
Regimen EI: (I) administration at the loading dose of (I-i) 3 mg/kg of the antibody once every week (weekly) for four weeks, (I-ii) 4.5 mg/kg of the antibody once every week (weekly) for two weeks, or (I-iii) 6 mg/kg of the antibody once every two weeks for four weeks, followed by (II) administration at the maintenance dose of (II-i) 1.5 mg/kg of the antibody once every week (weekly), (II-ii) 3 mg/kg of the antibody once every two weeks, or (II-iii) 6 mg/kg of the antibody once every four weeks (every month), followed by (III) administration at the first modified maintenance dose of (III-i) 2.25 mg/kg of the antibody once every week (weekly), (III-ii) 4.5 mg/kg of the antibody once every two weeks, or (III-iii) 9 mg/kg of the antibody once every four weeks (every month), followed by (IV) administration at the second modified maintenance dose of (IV-i) 4.5 mg/kg of the antibody once every week (weekly), or (IV-ii) 9 mg/kg of the antibody once every two weeks.
Regimen EJ: (I) administration at the loading dose of (I-i) 3 mg/kg of the antibody once every week (weekly) for four weeks, (I-ii) 4.5 mg/kg of the antibody once every week (weekly) for two weeks, or (I-iii) 6 mg/kg of the antibody once every two weeks for four weeks, followed by (II) administration at the maintenance dose of (II-i) 1.5 mg/kg of the antibody once every week (weekly), (II-ii) 3 mg/kg of the antibody once every two weeks, or (II-iii) 6 mg/kg of the antibody once every four weeks (every month), followed by (III) administration at the first modified maintenance dose of (III-i) 2.25 mg/kg of the antibody once every week (weekly), (III-ii) 4.5 mg/kg of the antibody once every two weeks, or (III-iii) 9 mg/kg of the antibody once every four weeks (every month), followed by (IV) administration at the second modified maintenance dose of (IV-i) 4.5 mg/kg of the antibody once every week (weekly), or (IV-ii) 9 mg/kg of the antibody once every two weeks, followed by (V) administration at the third modified maintenance dose of (V-i) 6 mg/kg of the antibody once every week (weekly), or (V-ii) 12 mg/kg of the antibody once every two weeks.
Regimen EK: (I) administration at the loading dose of (I-i) 3 mg/kg of the antibody once every week (weekly) for four weeks, (I-ii) 4.5 mg/kg of the antibody once every week (weekly) for two weeks, or (I-iii) 6 mg/kg of the antibody once every two weeks for four weeks, followed by (II) administration at the maintenance dose of (II-i) 1.5 mg/kg of the antibody once every week (weekly), (II-ii) 3 mg/kg of the antibody once every two weeks, or (II-iii) 6 mg/kg of the antibody once every four weeks (every month), followed by (III) administration at the first modified maintenance dose of (III-i) 3 mg/kg of the antibody once every week (weekly), (III-ii) 6 mg/kg of the antibody once every two weeks, or (III-iii) 12 mg/kg of the antibody once every four weeks (every month), followed by (IV) administration at the second modified maintenance dose of (IV-i) 4.5 mg/kg of the antibody once every week (weekly), or (IV-ii) 9 mg/kg of the antibody once every two weeks.
Regimen EL: (I) administration at the loading dose of (I-i) 3 mg/kg of the antibody once every week (weekly) for four weeks, (I-ii) 4.5 mg/kg of the antibody once every week (weekly) for two weeks, or (I-iii) 6 mg/kg of the antibody once every two weeks for four weeks, followed by (II) administration at the maintenance dose of (II-i) 1.5 mg/kg of the antibody once every week (weekly), (II-ii) 3 mg/kg of the antibody once every two weeks, or (II-iii) 6 mg/kg of the antibody once every four weeks (every month), followed by (III) administration at the first modified maintenance dose of (III-i) 3 mg/kg of the antibody once every week (weekly), (III-ii) 6 mg/kg of the antibody once every two weeks, or (III-iii) 12 mg/kg of the antibody once every four weeks (every month), followed by (IV) administration at the second modified maintenance dose of (IV-i) 4.5 mg/kg of the antibody once every week (weekly), or (IV-ii) 9 mg/kg of the antibody once every two weeks, followed by (V) administration at the third modified maintenance dose of (V-i) 6 mg/kg of the antibody once every week (weekly), or (V-ii) 12 mg/kg of the antibody once every two weeks.
Regimen EM: (I) administration at the loading dose of (I-i) 3 mg/kg of the antibody once every week (weekly) for four weeks, (I-ii) 4.5 mg/kg of the antibody once every week (weekly) for two weeks, or (I-iii) 6 mg/kg of the antibody once every two weeks for four weeks, followed by (II) administration at the maintenance dose of (II-i) 1.5 mg/kg of the antibody once every week (weekly), (II-ii) 3 mg/kg of the antibody once every two weeks, or (II-iii) 6 mg/kg of the antibody once every four weeks (every month), followed by (III) administration at the first modified maintenance dose of (III-i) 3 mg/kg of the antibody once every week (weekly), (III-ii) 6 mg/kg of the antibody once every two weeks, or (III-iii) 12 mg/kg of the antibody once every four weeks (every month), followed by (IV) administration at the second modified maintenance dose of (IV-i) 6 mg/kg of the antibody once every week (weekly), or (IV-ii) 12 mg/kg of the antibody once every two weeks.
Regimen EN: (I) administration at the loading dose of (I-i) 3 mg/kg of the antibody once every week (weekly) for four weeks, (I-ii) 4.5 mg/kg of the antibody once every week (weekly) for two weeks, or (I-iii) 6 mg/kg of the antibody once every two weeks for four weeks, followed by (II) administration at the maintenance dose of (II-i) 2.25 mg/kg of the antibody once every week (weekly), (II-ii) 4.5 mg/kg of the antibody once every two weeks, or (II-iii) 9 mg/kg of the antibody once every four weeks (every month).
Regimen EO: (I) administration at the loading dose of (I-i) 3 mg/kg of the antibody once every week (weekly) for four weeks, (I-ii) 4.5 mg/kg of the antibody once every week (weekly) for two weeks, or (I-iii) 6 mg/kg of the antibody once every two weeks for four weeks, followed by (II) administration at the maintenance dose of (II-i) 2.25 mg/kg of the antibody once every week (weekly), (II-ii) 4.5 mg/kg of the antibody once every two weeks, or (II-iii) 9 mg/kg of the antibody once every four weeks (every month), followed by (III) administration at the modified maintenance dose of (III-i) 3 mg/kg of the antibody once every week (weekly), (III-ii) 6 mg/kg of the antibody once every two weeks, or (III-iii) 12 mg/kg of the antibody once every four weeks (every month).
Regimen EP: (I) administration at the loading dose of (I-i) 3 mg/kg of the antibody once every week (weekly) for four weeks, (I-ii) 4.5 mg/kg of the antibody once every week (weekly) for two weeks, or (I-iii) 6 mg/kg of the antibody once every two weeks for four weeks, followed by (II) administration at the maintenance dose of (II-i) 2.25 mg/kg of the antibody once every week (weekly), (II-ii) 4.5 mg/kg of the antibody once every two weeks, or (II-iii) 9 mg/kg of the antibody once every four weeks (every month), followed by (III) administration at the first modified maintenance dose of (III-i) 3 mg/kg of the antibody once every week (weekly), (III-ii) 6 mg/kg of the antibody once every two weeks, or (III-iii) 12 mg/kg of the antibody once every four weeks (every month), followed by (IV) administration at the second modified maintenance dose of (IV-i) 4.5 mg/kg of the antibody once every week (weekly), or (IV-ii) 9 mg/kg of the antibody once every two weeks.
Regimen EQ: (I) administration at the loading dose of (I-i) 3 mg/kg of the antibody once every week (weekly) for four weeks, (I-ii) 4.5 mg/kg of the antibody once every week (weekly) for two weeks, or (I-iii) 6 mg/kg of the antibody once every two weeks for four weeks, followed by (II) administration at the maintenance dose of (II-i) 2.25 mg/kg of the antibody once every week (weekly), (II-ii) 4.5 mg/kg of the antibody once every two weeks, or (II-iii) 9 mg/kg of the antibody once every four weeks (every month), followed by (III) administration at the first modified maintenance dose of (III-i) 3 mg/kg of the antibody once every week (weekly), (III-ii) 6 mg/kg of the antibody once every two weeks, or (III-iii) 12 mg/kg of the antibody once every four weeks (every month), followed by (IV) administration at the second modified maintenance dose of (IV-i) 4.5 mg/kg of the antibody once every week (weekly), or (IV-ii) 9 mg/kg of the antibody once every two weeks, followed by (V) administration at the third modified maintenance dose of (V-i) 6 mg/kg of the antibody once every week (weekly), or (V-ii) 12 mg/kg of the antibody once every two weeks.
Regimen ER: (I) administration at the loading dose of (I-i) 3 mg/kg of the antibody once every week (weekly) for four weeks, (I-ii) 4.5 mg/kg of the antibody once every week (weekly) for two weeks, or (I-iii) 6 mg/kg of the antibody once every two weeks for four weeks, followed by (II) administration at the maintenance dose of (II-i) 2.25 mg/kg of the antibody once every week (weekly), (II-ii) 4.5 mg/kg of the antibody once every two weeks, or (II-iii) 9 mg/kg of the antibody once every four weeks (every month), followed by (III) administration at the first modified maintenance dose of (III-i) 3 mg/kg of the antibody once every week (weekly), (III-ii) 6 mg/kg of the antibody once every two weeks, or (III-iii) 12 mg/kg of the antibody once every four weeks (every month), followed by (IV) administration at the second modified maintenance dose of (IV-i) 6 mg/kg of the antibody once every week (weekly), or (IV-ii) 12 mg/kg of the antibody once every two weeks.
Regimen ES: (I) administration at the loading dose of (I-i) 3 mg/kg of the antibody once every week (weekly) for four weeks, (I-ii) 4.5 mg/kg of the antibody once every week (weekly) for two weeks, or (I-iii) 6 mg/kg of the antibody once every two weeks for four weeks, followed by (II) administration at the maintenance dose of (II-i) 2.25 mg/kg of the antibody once every week (weekly), (II-ii) 4.5 mg/kg of the antibody once every two weeks, or (II-iii) 9 mg/kg of the antibody once every four weeks (every month), followed by (III) administration at the modified maintenance dose of (III-i) 4.5 mg/kg of the antibody once every week (weekly), or (III-ii) 9 mg/kg of the antibody once every two weeks.
Regimen ET: (I) administration at the loading dose of (I-i) 3 mg/kg of the antibody once every week (weekly) for four weeks, (I-ii) 4.5 mg/kg of the antibody once every week (weekly) for two weeks, or (I-iii) 6 mg/kg of the antibody once every two weeks for four weeks, followed by (II) administration at the maintenance dose of (II-i) 2.25 mg/kg of the antibody once every week (weekly), (II-ii) 4.5 mg/kg of the antibody once every two weeks, or (II-iii) 9 mg/kg of the antibody once every four weeks (every month), followed by (III) administration at the first modified maintenance dose of (III-i) 4.5 mg/kg of the antibody once every week (weekly), or (III-ii) 9 mg/kg of the antibody once every two weeks, followed by (IV) administration at the second modified maintenance dose of (IV-i) 6 mg/kg of the antibody once every week (weekly), or (IV-ii) 12 mg/kg of the antibody once every two weeks.

In some embodiments, regimens of the invention can be applicable for patients who suffer from bleeding, or excessive bleeding. The regimens of the invention can be applicable in a method for preventing and/or treating bleeding in such patients, or for increasing blood clotting activity in such patients, or for reducing excessive bleeding in such patients. Here, “preventing” or “treating” bleeding refers to reducing the incidence of bleeding or reducing the likelihood of bleeding in a patient. In certain embodiments, excessive bleeding in such patients is caused by a decrease or deficiency in the activity of FVIII and/or FVIIIa. In a certain embodiment, patents who suffer from bleeding have hemophilia, which may be hemophilia A or severe hemophilia A.

In some embodiments, regimens of the invention can be applicable for patients with hemophilia A and preferably patients with hemophilia A having FVIII inhibitors and/or patients with hemophilia A not having FVIII inhibitors.

In some embodiment, regimens of the invention can be applicable for patients with severe hemophilia A.

In some embodiments, regimens of the invention can be applicable for adult patients, and/or pediatric patients and/or such special population of patients likely to exhibit lower exposure.

The dosage regimen is determined, for example, by considering the effects and safety. Furthermore, the dosage regimen is determined by considering the convenience of the patient, within the range that does not impair the effectiveness and safety. For example, the dosage regimen for a hemophilia A patient can be determined by considering the effects of preventing bleeding in patients and clinically acceptable safety.

A disease accompanying bleeding or a disease caused by bleeding may include a disease that develops and/or progresses due to a decrease or deficiency in the activity of FVIII and/or FVIIIa.

A disease that develops and/or progresses due to a decrease or deficiency in the activity of FVIII and/or FVIIIa is, for example, hemophilia A, hemophilia A with emergence of an inhibitor against FVIII/FVIIIa, acquired hemophilia A, and von Willebrand disease, but the disease is not particularly limited thereto.

In some embodiments, regimens of the invention can be applicable for preventing bleeding episodes and reducing the frequency of bleeding episodes in congenital FV111-deficient patients with inhibitors.

In some embodiments, regimens of the invention can be applicable for preventing bleeding episodes and reducing the frequency of bleeding episodes in congenital FVlll-deficient patients without inhibitors.

In some embodiments, regimens of the invention can be applicable for preventing bleeding episodes and reducing the frequency of bleeding episodes in acquired FVlll-deficient patients with inhibitors.

In some embodiments, regimens of the invention can be applicable for preventing bleeding episodes and reducing the frequency of bleeding episodes in acquired FVlll-deficient patients without inhibitors.

In some embodiments, regimens of the invention can be applicable for preventing bleeding episodes and reducing the frequency of bleeding episodes in congenital von Willebrand factor-deficient patients.

In some embodiments, regimens of the invention can be applicable for preventing bleeding episodes and reducing the frequency of bleeding episodes in acquired von Willebrand factor-deficient patients.

The term “inhibitor patient” as used herein refers to a patient with hemophilia A having FVIII inhibitors.

The term “non-inhibitor patient” as used herein refers to a patient with hemophilia A not having FVIII inhibitors.

The present invention provides a product comprising at least (i) a container; (ii) a pharmaceutical composition in a container, which comprises a bispecific antigen-binding molecule that recognizes (a) FIX and/or FIXa and (b) FX and/or FXa; and (iii) a document instructing administration of the antigen-binding molecule according to any one of the dosing regimens described above. In addition, a label, syringe, syringe needle, pharmaceutically acceptable medium, alcohol-soaked cotton, adhesive bandage, and such may be packaged in the product. The container is, for example, a bottle, a glass bottle, or a syringe, and can be produced from various materials such as glass or plastic. Administration-supporting devices may be attached to the product. A pharmaceutical composition is stored in the container, and the mouth of the container is sealed with a rubber stopper or such. For example, a label indicating that the pharmaceutical composition is to be used for preventing or treating selected pathological conditions is attached to the container. The document of (iii) may include instructions that specify the loading dose, maintenance dose, administration frequency or intervals, according to the dosing regimens as described above.

Treatment of hemophilia refers to, for example, stopping bleeding by administering the composition to a hemophilia patient who is actually showing bleeding symptoms (treatment of bleeding) and/or reducing the bleeding frequency by administering the composition to a patient who had shown bleeding to prevent manifestation of bleeding symptoms in advance (prevention of bleeding), but it is not limited thereto. Treatment and prevention of bleeding may be understood as having the same meaning in certain cases and such treatment and prevention of bleeding may be called prophylaxis therapy or regular administration therapy of a therapeutic agent (the bispecific antigen-binding molecules of the present invention).

Prevention of hemophilia refers to, for example, reducing the incidence of hemophilia or reducing the likelihood of hemophilia.

Herein, bleeding that is examined and counted as the number of bleeding events in a patient is, for example, bleeding that required hemostatic treatment by coagulation factor formulations. Coagulation factor formulations refer to, for example, FVIII formulations and bypassing agents (activated prothrombin complex formulations, recombinant FVIIa formulations, and such).

The number of bleedings per year (the Annualized Bleeding Rate (ABR)) is calculated as, for example: (number of bleeding events×365.25)/number of days of observation.

The present invention provides a pharmaceutical composition comprising a bispecific antigen-binding molecule which recognizes (a) FIX and/or FIXa and (b) FX and/or FXa, as a more effective pharmaceutical composition for preventing and/or treating bleeding, a disease accompanying bleeding, or a disease caused by bleeding, the disease including those that develops and/or progresses due to a decrease or deficiency in the activity of FVIII and/or FVIIIa, or a dosage regimen thereof.

All prior art references cited herein are incorporated by reference into this description.

### EXAMPLES

The present invention is specifically illustrated below with reference to Examples, but it is not construed as being limited thereto.

### Example 1

Efficacious Dosing Regimens with Administration Interval Extension and/or Dose Up-Titration Dose-Exposure-Response Model Development

A population pharmacokinetic (PopPK) model of emicizumab was developed using the quantifiable plasma emicizumab concentration data from 42 healthy subjects (NPL 8) and 18 patients with hemophilia A having or not having FVIII inhibitors (NPLs 9 and 10). In addition, the exposure-response relationship on the bleeding-prophylactic efficacy of emicizumab was quantitatively characterized based on a repeated time-to-event (RTTE) modeling approach using the bleeding onset data from the same 18 patients (NPLs 9 and 10).

A one-compartment model with first-order absorption and first-order elimination was employed as the structural PopPK model to describe the plasma emicizumab concentration-time profile.

The parameter estimates of the PopPK model including covariate effects are listed in Table 1.

TABLE 1
Parameter Estimates of the Population Pharmacokinetic Model

| | | |
|---|---|---|
| Parameter | Unit | Estimate^{a)} |

Model structure

| | | |
|---|---|---|
| CL/F^{b)} | L/day | 0.222 |
| V_{d}/F^{b)} | L | 10.2 |
| t_{1/2, abs} | day | 1.56 |

Covariate effect

| | | |
|---|---|---|
| Onset time of the effect of influential ADA on | day | 33.4 |
| CL/F after emicizumab administration | | |
| Effect of influential ADA on CL/F^{c)} | — | 2.01 |
| Effect of BW on CL/F^{d)} | — | 0.75 (fixed) |
| Effect of BW on V_{d}/F^{d)} | — | 1 (fixed) |
| Effect of PAT on CL/F^{c)} | — | 0.232 |
| Effect of PAT on V_{d}/F^{c)} | — | 0.175 |

Inter-individual variability^{e)}

| | | |
|---|---|---|
| Variance for CL/F | — | 0.0737 |
| Variance for V_{d}/F | — | 0.0455 |
| Variance for t_{1/2, abs} | — | 0.502 |
| Covariance for CL/F and V_{d}/F | — | 0.0278 |

Residual unexplained variability^{f)}

| | | |
|---|---|---|
| Additive error^{g)} | μg/mL | 0.0149 |
| Proportional error^{h)} | % | 12.8 |

CL/F: apparent total clearance, V_{d}/F: apparent volume of distribution, t_{1/2, abs}: first-order absorption half-life, ADA: influential anti-drug antibodies, BW: body weight, PAT: patient.
^{a)}estimated using NONMEM software version 7.2.0 (ICON Development Solutions, Ellicott City, MD, USA),
^{b)}standardized by a typical body weight of 70 kg,
^{c)}parameterized as log-transformed geometric mean ratio,
^{d)}parameterized as allometric exponent (assumed to work in a power manner),
^{e)}assumed to follow an exponential error model,
^{f)}assumed to follow a combined additive plus proportional error model,
^{g)}parameterized as standard deviation,
^{h)}parameterized as coefficient of variation.

RTTE modeling dealt with the bleeding events that occurred both before (i.e., for approximately 6 months) and after the start of emicizumab administration and required the on-demand use of coagulation factor products, regardless of the bleeding site. A time-varying hazard model as the structural RTTE model consisted of a constant baseline hazard (lambda) to account for the bleeding rate when treated with the on-demand therapy with coagulation factor products and an Eₘₐₓ model to account for the bleeding-prophylactic effect of emicizumab as a function of plasma emicizumab concentration. The model equation of the RTTE model is illustrated below, and the parameter estimates are listed in Table 2.

$$h ( t ) = λ \times ( 1 - \frac{\frac{{C}_{p} ( t )}{E {C}_{50}} + {E}_{PLX}}{1 + \frac{{C}_{p} ( t )}{E {C}_{50}} + {E}_{PLX}} ) \qquad [ Math . 1 ]$$

h(t): hazard on bleeding onset at a time of t, C_{P}(t): population pharmacokinetic model-predicted plasma emicizumab concentration at a time of t, λ(lambda): baseline hazard on bleeding onset (annual bleeding rate), EC₅₀: plasma emicizumab concentration to achieve a half of the maximum prophylactic effect on bleeding onset, E_{P LX}: effect of the prophylactic therapy with coagulation factor products when ongoing.

TABLE 2
Parameter Estimates of the Repeated Time-to-Event Model

| | | |
|---|---|---|
| Parameter | Unit | Estimate^{a)} |

Model structure

| | | |
|---|---|---|
| λ (lambda) | count/year | 21.9 |
| EC₅₀ | μg/mL | 1.19 |
| E_{PLX} | — | 0.314 |

Inter-individual variability^{b)}

| | | |
|---|---|---|
| Variance for λ | — | 0.340 |
| Variance for EC₅₀ | — | 2.53 |

lambda: baseline hazard on bleeding onset (annual bleeding rate), EC₅₀: plasma emicizumab concentration to achieve a half of the maximum prophylactic effect on bleeding onset, E_{PLX}: effect of the prophylactic therapy with coagulation factor products when ongoing.
^{a)}estimated using NONMEM software version 7.2.0 (ICON Development Solutions, Ellicott City, MD, USA),
^{b)}assumed to follow an exponential error model.

Determination of Efficacious Exposure and Justification of Dose Up-Titration

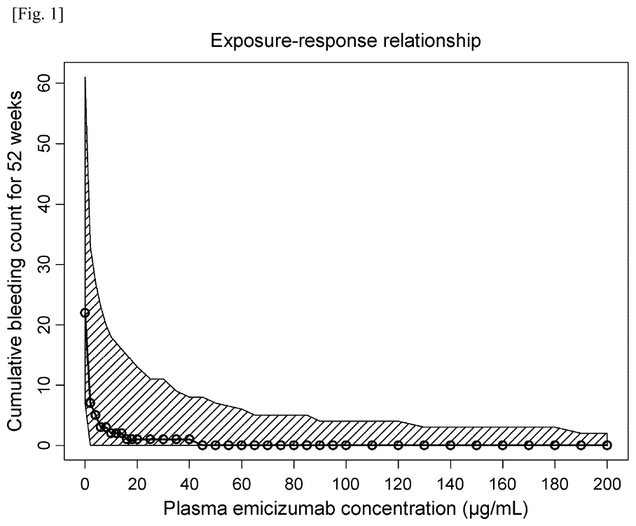

The RTTE model-simulated relationship between annual bleeding rate (ABR) and plasma emicizumab concentration is shown in FIG. 1. A plasma emicizumab concentration of 45 micro g/mL or more is expected to achieve a median ABR of zero indicating no bleeding onset for a year in 50% or more of patients.

Further improvement in bleeding-prophylactic efficacy outcome is not expected at plasma emicizumab concentrations of 45 micro g/mL or more in terms of median ABR. However, due to the inter-individual variability in drug effect and baseline ABR, there would remain considerable proportions of patients with ABR of >0 even at such plasma emicizumab concentrations. Therefore, it is likely that increasing plasma emicizumab concentration (emicizumab dose) results in further reduced ABR in patients with imperfect bleeding control. In addition, in case of a special population likely to exhibit lower exposure (e.g., pediatric patients), increasing emicizumab dose should make sense to provide increased plasma emicizumab concentration and accordingly further reduced ABR.

Determination of Efficacious Dose and Justification of Administration Interval Extension

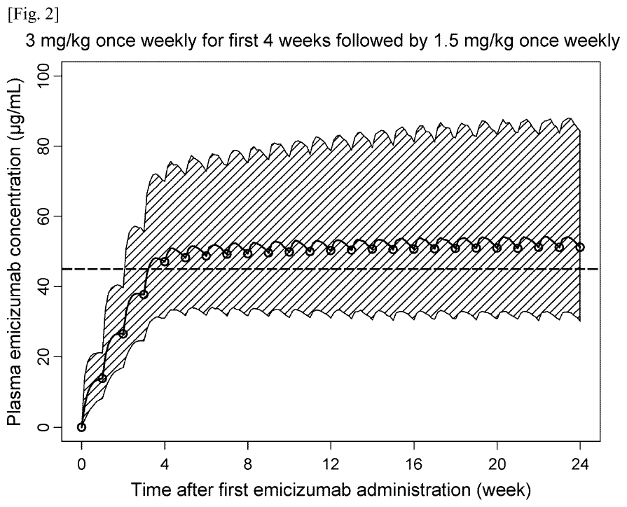

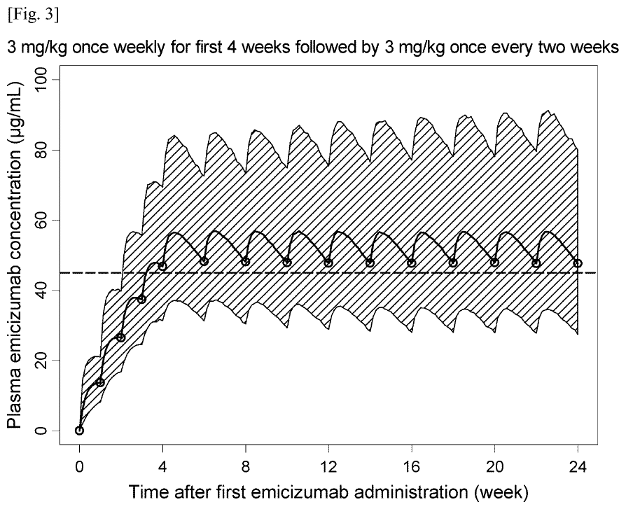

As doses to achieve the target exposure level of 45 micro g/mL, once-weekly loading dose of 3 mg/kg for the first 4 weeks followed by once-weekly maintenance dose of 1.5 mg/kg or once-every-two-week maintenance dose of 3 mg/kg were proposed. The repeated loading doses were intended to achieve the steady-state plasma concentration as early as possible. The PopPK model-simulated plasma emicizumab concentration-time profiles at the proposed dosing regimens are shown in FIG. 2 and FIG. 3. The simulations indicated that more than half of patients should achieve the target exposure level of 45 micro g/mL at steady state (i.e., after 4 weeks onwards) at both dosing regimens.

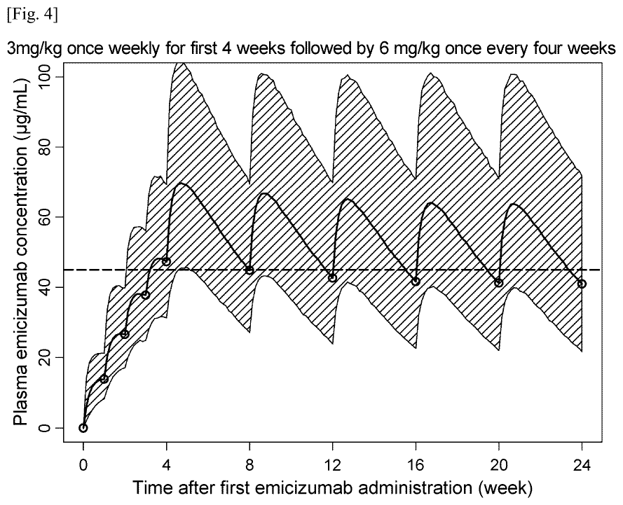

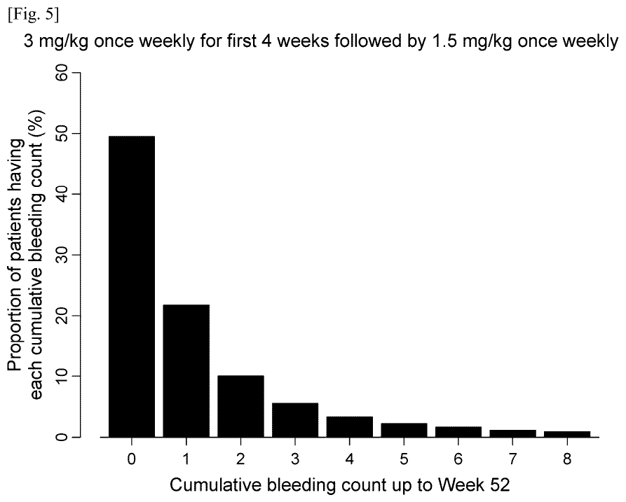

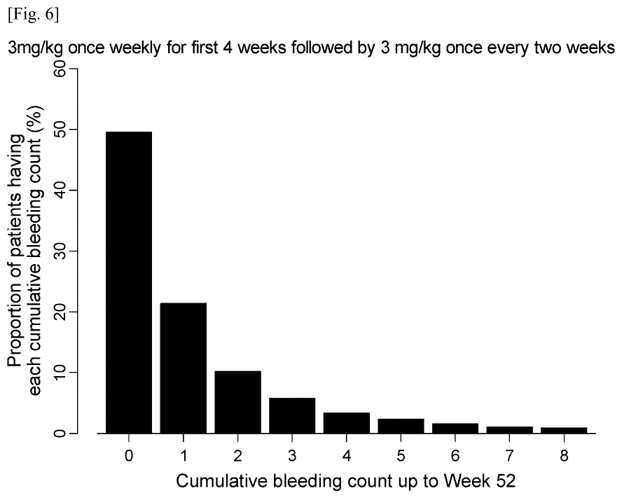

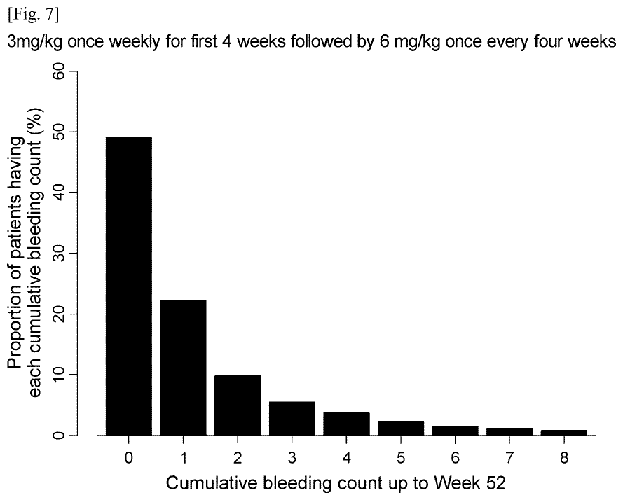

Another proposed dosing regimen of once-weekly loading dose of 3 mg/kg for the first 4 weeks followed by once-4-weekly maintenance dose of 6 mg/kg was not predicted to achieve the target exposure level of 45 micro g/mL as a median steady-state trough level, while providing a higher peak level due to a larger peak-trough fluctuation (FIG. 4). However, in terms of efficacy, the PopPK/RTTE model-simulated ABR distributions were predicted to be similar among the dosing regimens (FIG. 5, FIG. 6, and FIG. 7). This suggests that with a given dose per administration for maintenance dose which is higher than that for loading dose, extending the administration interval would result in a similar bleeding-prophylactic efficacy to other dosing regimens with a per-administration maintenance dose lower than or equal to the loading dose.

### Example 2

A Randomized, Multi-Center, Open-Label Phase III Clinical Trial to Evaluate the Efficacy, Safety and Pharmacokinetics of Prophylactic Emicizumab Versus No Prophylaxis in Hemophilia A Patients With Inhibitors (HAVEN 1)

This multicenter, open-label study evaluated the safety, efficacy and pharmacokinetics of prophylactic emicizumab treatment or regular emicizumab administration therapy in patients previously treated with episodic or prophylactic bypassing agents. Episodic bypassing agent patients were randomized in a 2:1 fashion to receive emicizumab prophylaxis (Arm A) versus no prophylaxis (Arm B) and were stratified across Arms A and B according to the number of bleeds they had experienced over the last 24 weeks prior to study entry (less than [<] 9 or greater than or equal to [>=] 9 bleeds); Arm B patients had the opportunity to switch to emicizumab prophylaxis after 24 weeks on-study. Prophylactic bypassing agent patients switched to emicizumab prophylaxis (Arm C) from the start of the trial; enrollment was extended for 24 weeks after the last patient enrolled in Arms A or B or until approximately 50 patients enrolled in Arm C, whichever occurred first. Episodic or prophylactic bypassing agent patients who previously participated in a Non-Interventional Study (NIS) BH29768 but were unable to enroll in Arms A or B prior to their closure had the opportunity to enroll in Arm D until 24 weeks after the last patient enrolled in Arms A or B or until approximately 35 patients enrolled in Arm D, whichever occurred first. Like patients in Arms A and C, Arm D patients received emicizumab prophylaxis from the start of the trial. All patients continued to receive standard of care/background treatment with their usual episodic bypassing agent therapy to treat breakthrough bleeds, as needed.
Arm A: Episodic Treatment+Study Drug (Emicizumab Prophylaxis)
Patients with inhibitors (patients that meet the inclusion criteria of diagnosis of congenital hemophilia A of any severity and documented history of high-titer inhibitor (i.e., 5 or more Bethesda Units [BU])) who received episodic treatment with bypassing agents prior to study entry were randomized to receive emicizumab prophylaxis when they start the trial.
Arm B: Episodic Treatment (No Prophylaxis)
Patients with inhibitors who received episodic treatment with bypassing agents prior to study entry were randomized to continue episodic bypassing agent treatment when they start the trial; they had the opportunity to switch to emicizumab prophylaxis after 24 weeks on-study.
Arm C: Study Drug (Emicizumab Prophylaxis)+Episodic Treatment
Patients with inhibitors who received prophylactic treatment with bypassing agents prior to study entry received emicizumab prophylaxis when they start the trial.
Arm D: Study Drug (Emicizumab Prophylaxis)+Episodic Treatment
Episodic or prophylactic bypassing agent patients who previously participated in a Non-Interventional Study BH29768 but were unable to enroll in Arms A or B prior to their closure had the opportunity to enroll in Arm D, in which they received emicizumab prophylaxis when they started the trial.

Emicizumab was administered subcutaneously at a dose of 3 mg/kg/week for 4 weeks followed by 1.5 mg/kg/week up to the end of the study.

The primary endpoint was the reduction of frequency in bleeding for patients who received subcutaneously administered emicizumab at a dose of 3 mg/kg/week for 4 weeks followed by 1.5 mg/kg/week compared to patients without the drug. The number of bleeds was significantly reduced in patients with the drug as described in more detail below.

Study Population

One hundred and nine participants were enrolled. All participants were male and had a median age of 28 years (range, 12-75; Table 3); the median age in Arm C was lower, which was consistent with higher prior use of prophylactic BPAs in this younger group. While most had severe hemophilia, 7/109 participants had mild or moderate disease. Approximately 40% of participants in Arms A, B, and D received prior immune tolerance induction (ITI), while 67% of participants in Arm C previously underwent ITI. The majority of participants (69.7%) had target joints, with 69.7% having >1 target joint. Median (range) emicizumab treatment exposure was 24.0 weeks (3.0-47.9) overall. Median (range) duration of emicizumab treatment exposure were Arm A, 29.5 (3.3-47.9) weeks; Arm B, 8.0 (4.0-16.0) weeks; Arm C, 19.0 (5.9-45.0) weeks and Arm D, 5.8 (3.0-14.0) weeks.

TABLE 3-1
Participant demographics and baseline characteristics

| Study arm | | | | |
|---|---|---|---|---|
| A: | B: | C: | D: | |
| Emicizumab | No | Emicizumab | Emicizumab | |
| prophylaxis | prophylaxis | prophylaxis | prophylaxis | Total |
| (N = 35) | (N = 18) | (N = 49) | (N = 7) | (N = 109) |

Sex, n (%)

| | | | | | |
|---|---|---|---|---|---|
| Male | 35 (100) | 18 (100) | 49 (100) | 7 (100) | 109 (100) |

Age, years

| | | | | | |
|---|---|---|---|---|---|
| Median (min-max) | 38.0 (12-68) | 35.5 (13-65) | 17.0 (12-75) | 26.0 (19-49) | 28.0 (12-75) |

Age, n (%)

| | | | | | |
|---|---|---|---|---|---|
| <18 years | 4 (11.4) | 2 (11.1) | 26 (53.1) | 0 (0) | 32 (29.4) |
| ≥18 years | 31 (88.6) | 16 (88.9) | 23 (46.9) | 7 (100) | 77 (70.6) |

Hemophilia severity at baseline, n (%)

| | | | | | |
|---|---|---|---|---|---|
| Mild | 2 (5.7) | 0 (0) | 1 (2.0) | 0 (0) | 3 (2.8) |
| Moderate | 2 (5.7) | 0 (0) | 1 (2.0) | 1 (14.3) | 4 (3.7) |
| Severe | 31 (88.6) | 18 (100) | 47 (95.9) | 6 (85.7) | 102 (93.6) |

Bleeding events in 24 weeks prior to study entry, n (%)

| | | | | | |
|---|---|---|---|---|---|
| <9 | 11 (31.4) | 5 (27.8) | 23 (46.9) | 4 (57.1) | 43 (39.4) |
| ≥9 | 24 (68.6) | 13 (72.2) | 26 (53.1) | 3 (42.9) | 66 (60.6) |

Target joints,* n (%)

| | | | | | |
|---|---|---|---|---|---|
| No | 10 (28.6) | 5 (27.8) | 15 (30.6) | 3 (42.9) | 33 (30.3) |
| Yes | 25 (71.4) | 13 (72.2) | 34 (69.4) | 4 (57.1) | 76 (69.7) |
| 1 | 7 (28.0) | 3 (23.1) | 10 (29.4) | 3 (75.0) | 23 (30.3) |
| >1 | 18 (72.0) | 10 (76.9) | 24 (70.6) | 1 (25.0) | 53 (69.7) |

Highest historical inhibitor titer (BU)

| | | | | | |
|---|---|---|---|---|---|
| n | 32 | 16 | 47 | 6 | 101 |
| Mean (SD) | 288.9 (472.8) | 706.8 (1450.0) | 815.7 (1148.1) | 528.9 (793.4) | 614.5 (1037.9) |
| Median | 84.5 | 102.0 | 309.0 | 240.0 | 180.0 |
| Min-Max | 5-1570 | 18-4500 | 11-5000 | 28-2125 | 5-5000 |
| <5 BU, n/N (%) | 0/35 (0) | 0/18 (0) | 0/49 (0) | 0/7 (0) | 0/109 (0) |
| ≥5 BU, n/N (%) | 32/35 (91.4) | 16/18 (88.9) | 47/49 (95.9) | 6/7 (85.7) | 101/109 (92.7) |
| Unknown, n/N | 3/35 (8.6) | 2/18 (11.1) | 2/49 (4.1) | 1/7 (14.3) | 8/109 (7.3) |
| (%) | | | | | |

TABLE 3-2
Episodic coagulation product use in 24 weeks prior to study entry, n (%)

| | | | | | |
|---|---|---|---|---|---|
| Any products | 35 (100) | 18 (100) | 23 (47) | 7 (100) | 83 (76) |
| aPCC | 27 (77.1) | 13 (72.2) | 15 (65.2) | 5 (71.4) | 60 (72.3) |
| rFVIIa | 22 (62.9) | 17 (94.4) | 15 (65.2) | 5 (71.4) | 59 (71.1) |
| Factor VIII | 1 (2.9) | 0 (0) | 1 (4.3) | 2 (28.6) | 4 (4.8) |
| Other | 1 (2.9) | 0 (0) | 0 (0) | 1 (14.3) | 2 (2.4) |

Prophylactic coagulation product use in 24 weeks prior to study entry, n (%)

| | | | | | |
|---|---|---|---|---|---|
| Any products | 0 (0) | 0 (0) | 49 (100) | 0 (0) | 49 (45) |
| aPCC | 0 (0) | 0 (0) | 36 (73.5) | 0 (0) | 36 (73.5) |
| rFVIIa | 0 (0) | 0 (0) | 15 (30.6) | 0 (0) | 15 (30.6) |
| Factor VIII | 0 (0) | 0 (0) | 1 (2.0) | 0 (0) | 1 (2.0) |
| Other | 0 (0) | 0 (0) | 1 (2.0) | 0 (0) | 1 (2.0) |

*% based on number of participants with target joints, all numbers are based on eCRF and not NIS data.
aPCC, activated prothrombin complex concentrates; NIS, non-interventional study; rFVIIa, recombinant factor VIIa.

Efficacy

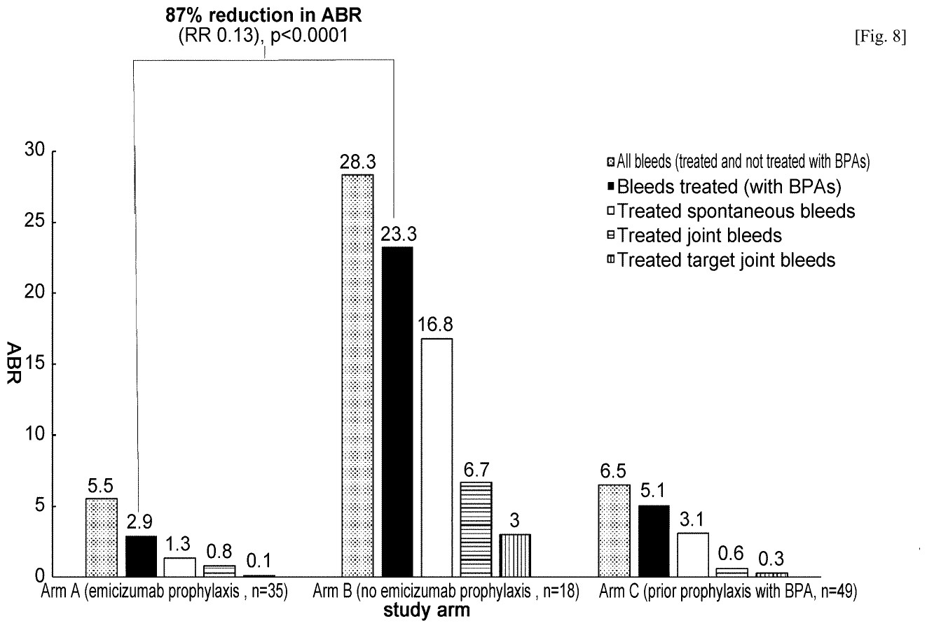

There was a statistically significant and clinically meaningful 87% reduction in bleed rates between Arms A (emicizumab prophylaxis) versus B (no prophylaxis); ABR (95% CI) 2.9 (1.69; 5.02) versus 23.3 (12.33; 43.89), p<0.0001 (FIG. 8 and Table 4). Statistically significant and clinically meaningful reductions were also observed in all secondary bleed-related endpoints, including spontaneous, joint, and target joint bleeds, and all bleeds. Overall, 62.9% (22/35) of participants randomized to emicizumab prophylaxis experienced zero bleeds (FIG. 8 and Table 4).

TABLE 4
Bleeds across study arms

| | Study arms | | |
|---|---|---|---|
| | A: | B: | C: |
| | Emicizumab | No | Emicizumab |
| | prophylaxis | prophylaxis | prophylaxis |
| Bleeds | (N = 35) | (N = 18) | (N = 49) |
| Treated bleeds (with BPAs) | | | |
| ABR, model based^{a} (95% CI) | 2.9 (1.69; 5.02) | 23.3 (12.33; 43.89) | 5.1 (2.28; 11.22) |
| % reduction (RR), p value | 87% (0.13), p < 0.0001 | | — |
| Median ABR, calculated (IQR) | 0.0 (0.0; 3.7) | 18.8 (13.0; 35.1) | 0.0 (0.0; 1.7) |
| All bleeds (treated/not treated with BPAs) | | | |
| ABR, model based^{a} (95% CI) | 5.5 (3.58; 8.60) | 28.3 (16.79; 47.76) | 6.5 (3.43; 12.43) |
| % reduction (RR), p value | 80% (0.20), p < 0.0001 | | — |
| Median ABR, calculated (IQR) | 2.0 (0.0; 9.9) | 30.2 (18.3; 39.4) | 0.0 (0.0; 6.0) |
| Treated spontaneous bleeds | | | |
| ABR, model based^{a} (95% CI) | 1.3 (0.73: 2.19) | 16.8 (9.94; 28.30) | 3.1 (1.20; 8.02) |
| % reduction (RR), p value | 92% (0.08), p < 0.0001 | | — |
| Median ABR, calculated (IQR) | 0.0 (0.0; 3.3) | 15.2 (6.6; 30.4) | 0.0 (0.0; 0.0) |
| Treated joint bleeds | | | |
| ABR, model based^{a} (95% CI) | 0.8 (0.26; 2.20) | 6.7 (1.99; 22.42) | 0.6 (0.21; 1.48) |
| % reduction (RR), p value | 89% (0.11), 0.0050 | | — |
| Median ABR, calculated (IQR) | 0.0 (0.0; 0.0) | 1.0 (0.0; 14.4) | 0.0 (0.0; 0.0) |
| Treated target joint bleeds | | | |
| ABR, model based^{a} (95% CI) | 0.1 (0.03; 0.58) | 3.0 (0.96; 9.13) | 0.3 (0.10; 0.95) |
| % reduction (RR), p value | 95% (0.05), 0.0002 | | — |
| Median ABR, calculated (IQR) | 0.0 (0.0; 0.0) | 1.0 (0.0; 6.5) | 0.0 (0.0; 0.0) |
| Participants with zero bleeds, % (95% CI) | 62.9 (44.9; 78.5) | 5.6 (0.1; 27.3) | 69.4 (54.6; 81.7) |

^{a}Negative binomial model.
ABR, annualized bleeding rate; BPA, bypassing agent; CI, confidence interval; IQR, interquartile range; RR, relative risk

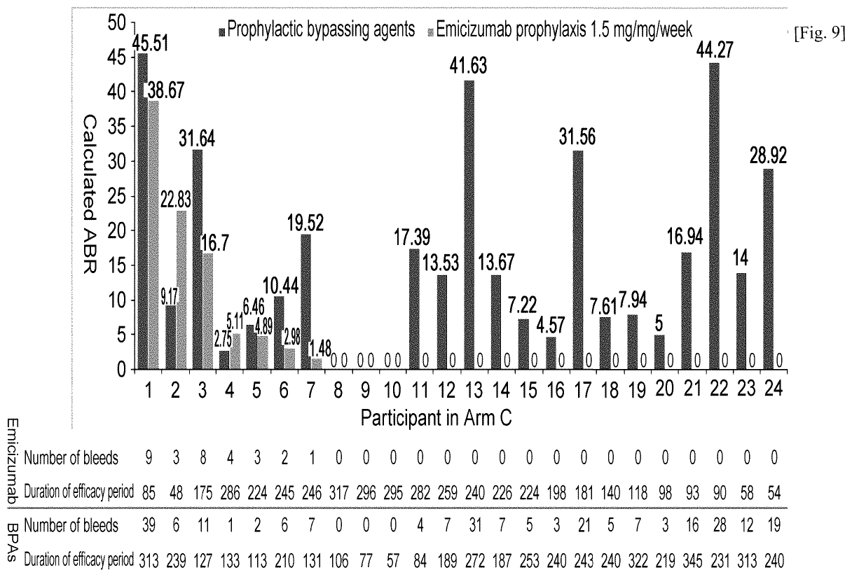

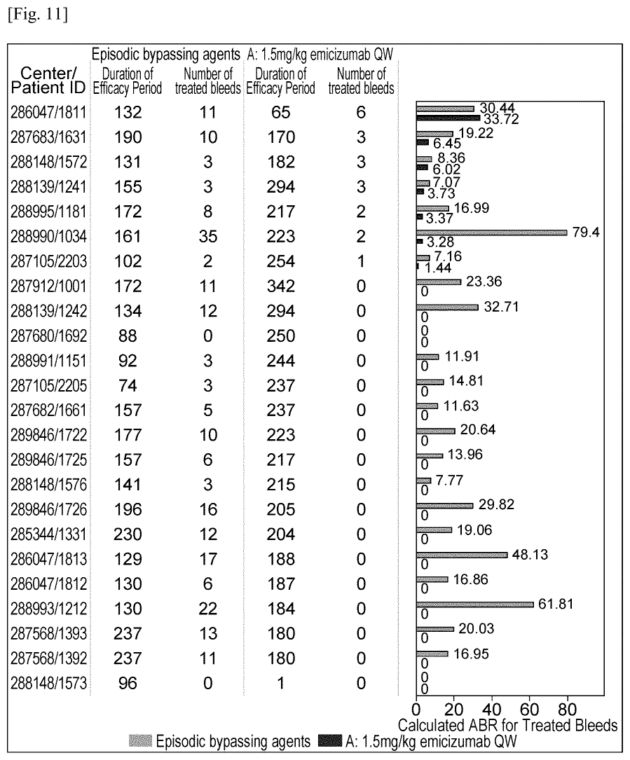

The intra-participant comparison in those who had previously participated in the NIS showed that emicizumab prophylaxis significantly reduced bleed rate versus previous BPA prophylaxis (79%: RR 0.21; p=0.0003 [Arm C]); individual participant data are shown in FIG. 9 and corresponding data for the intra-participant comparison versus prior episodic BPAs (Arm A) are shown in FIG. 11. Intra-participant comparison for emicizumab prophylaxis versus prior episodic BPA treatment showed a significant reduction in the risk of treated bleeds (92%: RR 0.08; p<0.0001 [Arm A]).

Emicizumab prophylaxis was associated with statistically significant and clinically meaningful improvements in health-related quality of life (HRQoL) and health status compared with no prophylaxis. Differences in adjusted means observed in study and clinically important differences determined from published literature (Wyrwich et al. Interpreting important health-related quality of life change using the Haem-A-QoL. Haemophilia. 2015;21(5):578-584.; Walters and Brazier. Comparison of the minimally important difference for two health state utility measures: EQ-5D and SF-6D. Qual Life Res. 2005;14(6):1523-1532.; Pickard et al. Estimation of minimally important differences in EQ-5D utility and VAS scores in cancer. Health Qual Life Outcomes. 2007;5:70.), respectively, were as follows: Haem-A-QoL physical health subscale, 21.55 (p=0.0029) and 10 points; Haem-A-QoL total score, 14.01 (p=0.0019) and 7 points; EQ-5D-5L VAS, −9.72 (p=0.0171) and 7 points; and, EQ-5D-5L Index utility score, −0.16 (p=0.0014) and 0.07 points.

Pharmacokinetic and Pharmacodynamic Outcomes

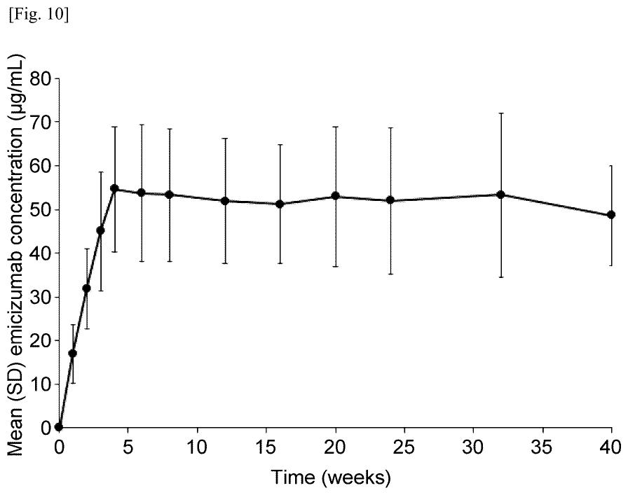

Mean trough emicizumab concentrations of >50 micro g/mL in blood were achieved after 4 weeks with administrations of the loading dose of 3 mg/kg/week, and sustained over the course of the study with administrations of the maintenance dose of 1.5 mg/kg/week (FIG. 10). D-dimer and prothrombin fragment 1.2 were not affected by emicizumab treatment.

### Example 3

Efficacy, safety and pharmacokinetics (PK) of Once-Weekly Prophylactic (Px) Emicizumab (ACE910) in Pediatric (<12 years) Persons with Hemophilia A with Inhibitors (PwHAwI): Interim Analysis of Single-Arm, Multicenter, Open-Label, Phase 3 Study (HAVEN 2)

The study enrolled PwHAwI aged <12 years (or 12-17 years if <40 kg) previously treated with bypassing agents (BPAs) to receive emicizumab prophylaxis for 52 or more weeks. Efficacy objectives included annualized bleeding rate (ABR) and bleed reduction versus historical bleed rate (non-interventional study).

Participants received weekly subcutaneous (SC) administrations of emicizumab for a designated period of 52 weeks. All participants continued to receive standard of care/background treatment with their usual episodic bypassing agent therapy to treat breakthrough bleeds as needed.

Emicizumab was administered SC once weekly for 52 weeks, 3 milligrams per kilogram per week (mg/kg/week) for 4 weeks and 1.5 mg/kg/week thereafter. The regimen was to be adapted based upon efficacy/bleed control.

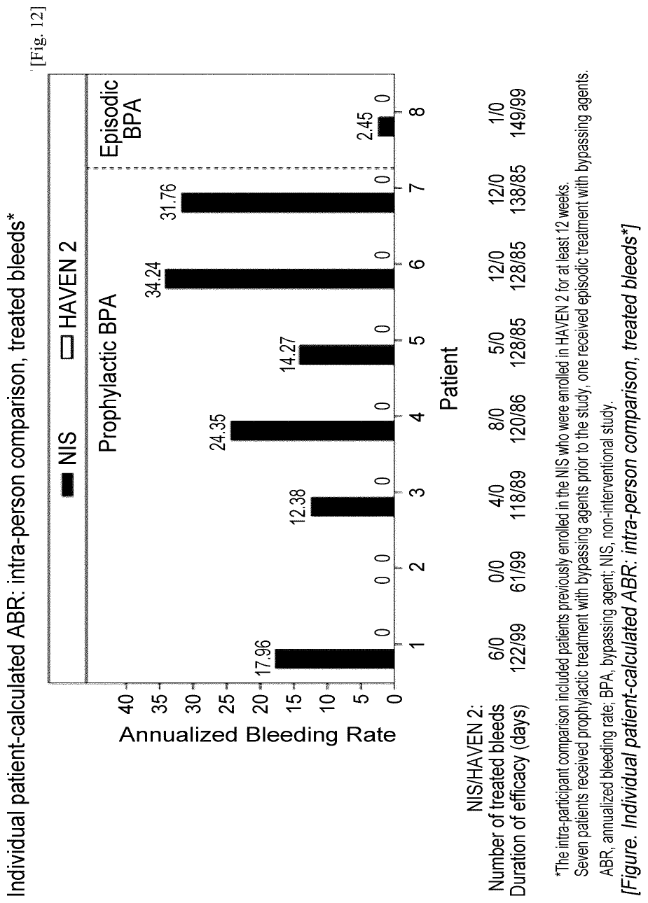

Interim analysis included 20 PwHAwI aged 3-12 years (median 8.5); 19 aged <12 years were included in the efficacy analyses (Table 5). The median observation time was 12.1 weeks (range 7.1-14.1). In total, 18/19 (94.7%) PwHAwI had zero treated bleeds and 12/19 (63.2%) did not bleed while on study. Overall, 14 bleeds were reported in 7 PwHAwI; with none occurring in a joint or muscle. A substantial reduction in ABR on study versus ABR from the NIS was observed in 8 PwHAwI included in the intra-participant comparison (FIG. 12); 8/8 had 100% reduction in number of treated bleeds, 5/8 had 100% reduction in number of all bleeds, and all PwHAwI had >75% reduction of all bleeds. Mean trough emicizumab concentrations of >50 micro g/mL were achieved after 4 weeks and sustained.

Emicizumab Px (prophylaxis) was safe and prevented/reduced bleeds in pediatric PwHAwI, showing clinically meaningful reductions in ABR compared with historical ABR. PK was similar to that seen in adult PwHA (persons with hemophilia A). These interim data show the potential for emicizumab to reduce the treatment and disease burden for pediatric PwHAwI.

TABLE 5
Bleeding events in HAVEN 2 study

| | Mean ABR^{b} | % PwHAwI with zero bleeds |
|---|---|---|
| Bleeds^{a} | (95% CI) N = 10 | (95% CI) N = 19 |
| Treated bleeds | 0.4 (0.00; 4.51) | 94.7 (74.0; 99.9) |
| All bleeds | 3.7 (0.94; 9.81) | 63.2 (38.4; 83.7) |
| Treated spontaneous | 0.4 (0.00; 4.51) | 94.7 (74.0; 99.9) |
| bleeds | | |

^{a}Bleed/medication questionnaire completed by caregiver via an electronic handheld device. Bleed definitions based on ISTH criteria (Blanchette VS, et al. J Thromb Haemost 2014; 12: 1935-39). Treated bleeds: bleeds treated with BPAs. All bleeds: bleeds treated and not treated with BPAs.
^{b}ABR calculated for persons on HAVEN 2 study 12 or more weeks.

### Example 4

A Randomized, Multi-Center, Open-Label, Phase 3 Clinical Study in Participants Aged 12 Years or Older to Evaluate the Efficacy, Safety, and Pharmacokinetics of Prophylactic Emicizumab Versus No Prophylaxis in Participants with Severe Hemophilia A Without Inhibitors against FVIII (HAVEN 3)

This is a randomized, multicenter, open-label, Phase 3 clinical study in participants aged 12 years or older to evaluate the efficacy, safety, and pharmacokinetics of prophylactic emicizumab versus no prophylaxis in participants with severe hemophilia A without inhibitors against FVIII (HAVEN 3).

Participants received emicizumab prophylaxis at the specified dose subcutaneously until the end of the study (maximum up to 2 years).
Experimental: Emicizumab (pre-study FVIII prophylaxis)
Participants who received FVIII prophylaxis prior to study entry received emicizumab prophylaxis at a dose of 3 mg/kg/week subcutaneously for 4 weeks, followed by 1.5 milligram per kilogram per week (mg/kg/week) emicizumab subcutaneously until the end of study (maximum up to 2 years).
Experimental: Emicizumab 1.5 mg/kg/week
Participants who received episodic treatment with FVIII prior to study entry received emicizumab prophylaxis at a dose of 3 mg/kg/week subcutaneously for 4 weeks, followed by 1.5 mg/kg/week emicizumab subcutaneously until the end of study (maximum up to 2 years).
Experimental: Emicizumab 3 mg/kg/2 weeks
Participants who received episodic treatment with FVIII prior to study entry received emicizumab prophylaxis at a dose of 3 mg/kg/week subcutaneously for 4 weeks, followed by 3 mg/kg once every 2 weeks (3 mg/kg/2 weeks) emicizumab subcutaneously until the end of study (maximum up to 2 years).
Active Comparator: No prophylaxis
Participants who received episodic treatment with FVIII prior to study entry were randomized to continue episodic FVIII treatment when they started the trial; they had the opportunity to switch to emicizumab prophylaxis after 24 weeks on-study.
Primary Outcome Measures:
Number of bleeds over time
Secondary Outcome Measures:
Reduction in number of bleeds over time [ Time Frame: Baseline, 24 weeks ]
Reduction in number of joint bleeds over time [ Time Frame: Baseline, 24 weeks ]
Reduction in number of target joint bleeds over time [ Time Frame: Baseline, 24 weeks ]
Health related quality of life scores [ Time Frame: 24 weeks ]
Trough plasma concentration (Ctrough) of emicizumab [ Time Frame: (Pre-dose) Every week during Weeks 1-4, every 2 weeks during Weeks 5-8, every 4 weeks during Weeks 9-24, every 8 weeks during Weeks 25-48, every 12 weeks thereafter (maximum up to 2 years) ]

### Example 5

A Multicenter, Open-Label, Non-Randomized Study to Assess the Efficacy, Safety, Pharmacokinetics, and Pharmacodynamics of Emicizumab Administered at a Dose of 6 milligrams per kilogram (mg/kg) Every 4 Weeks in Participants With Hemophilia A With or Without Inhibitors Against FVIII (HAVEN 4)

This multicenter, open-label, non-randomized study has assessed the efficacy, safety, pharmacokinetics, and pharmacodynamics of emicizumab administered at a dose of 6 milligrams per kilogram (mg/kg) once every 4 weeks (Q4W) in participants with hemophilia A with or without inhibitors against factor VIII (FVIII). The study consisted of 2 parts: a pharmacokinetic (PK) run-in part followed by an expansion part.

Emicizumab was administered according to the dose and schedule described in respective arms.
Experimental: Emicizumab: Expansion Part
Participants received emicizumab at a loading dose of 3 mg/kg once every week subcutaneously for initial 4 weeks followed by a maintenance dose of 6 mg/kg once every 4 weeks subcutaneously for a minimum of 24 weeks.
Experimental: Emicizumab: PK Run-in Part
Participants received emicizumab at a dose of 6 mg/kg once every 4 weeks subcutaneously for a minimum of 24 weeks.
Primary Outcome Measures:
Expansion Part: Number of Bleeding Events Over Time [ Time Frame: Expansion Part: Day 1 up to study completion (a minimum of 24 weeks, up to approximately 20 months)]

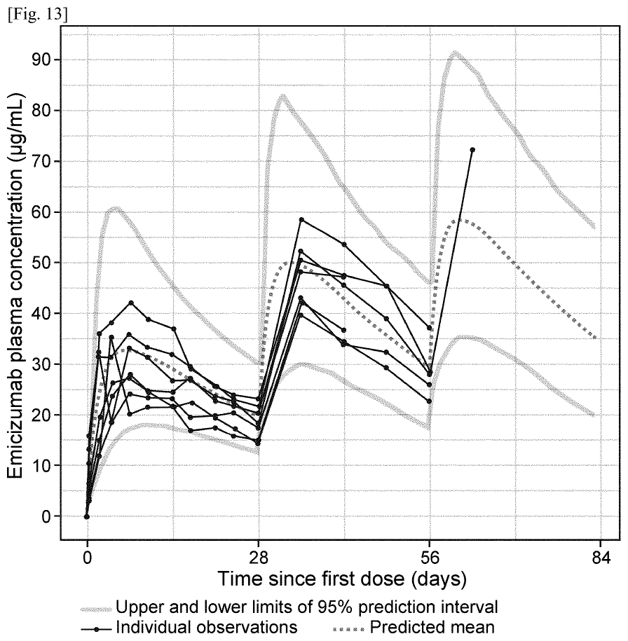

At the data cutoff of Apr. 10, 2017, 7 patients with severe hemophilia A had enrolled into the PK run-in cohort, 4 patients without inhibitors and 3 patients with inhibitors, of which 6 patients were aged 18 and over, and followed for a minimum of 6 weeks. Individual observed PK profiles were within the 95% prediction interval computed from a population PK model based on clinical data from a 1.5 mg/kg QW regimen (FIG. 13: gray bold solid lines indicate the upper and lower limits of the 95% prediction interval). Emicizumab PK parameters derived after a single SC administration of 6 mg/kg emicizumab (Table 6) were consistent with the values observed in previous studies with emicizumab (Uchida et al. Blood 2016; 127 (13):1633-1641). During the observation period (median, 8 weeks), 14 adverse events (AEs) were reported in 5 patients at the time of data cut-off, including 1 Grade 3 serious AE (worsening of hypertension); no AEs were considered related to the study drug. No anti-drug antibodies were detected. Also, 6 out of 7 patients had no bleeds while receiving Q4W emicizumab; 1 patient experienced 3 spontaneous nose bleeds on Study Days 12, 14, and 21, which did not require treatment.

TABLE 6
Summary of PK parameters after single
SC dose of 6 mg/kg emicizumab

| | Tₘₐₓ | Cₘₐₓ | AUC_{Day 28} | T_{1/2} |
|---|---|---|---|---|
| | (day) | (μg/mL) | (day*μg/mL) | (day) |
| Value* | 6.95 (3.99-7.18) | 31.8 (19.3) | 662.5 (19.7) | 30.2 (35.8) |

*Median (range) for Tₘₐₓ and geometric mean (CV %) for all other parameters.

Preliminary data from the HAVEN 4 study showed that Q4W dosing of emicizumab at 6 mg/kg per administration exhibited a PK behavior that was consistent with prior predictions, leading to an expected steady-state concentration average similar to the clinically confirmed dosing regimen (i.e., 1.5 mg/kg/QW). The safety and efficacy results from this PK run-in cohort enabled the opening of the HAVEN 4 expansion cohort, and provided promising support for a Q4W emicizumab prophylaxis regimen for the management of hemophilia A. The HAVEN 4 study is fully enrolled (N=48, including the PK run-in cohort patients).

### Example 6

A Multicenter, Open-Label, Phase III Clinical Trial to Evaluate the Efficacy, Safety, and Pharmacokinetics of Subcutaneous Administration of Emicizumab in Hemophilia A Pediatric Patients with Inhibitors

The study protocol of HAVEN 2 (see Example 3) has been amended to evaluate additional two emicizumab dosing schedules (once every 2 weeks [Q2W] and once every 4 weeks [Q4W]) as well as the originally planned dosing schedule (once weekly [QW]).

Overall, this non-randomized, multicenter, open-label, Phase III clinical study enrolls children with hemophilia A who have inhibitors against FVIII. Children with hemophilia A and documented historical FVIII inhibitor titer (5 BU or more) must currently be receiving treatment with bypassing agents. The study enrolls at least 40 patients younger than 12 years of age and up to approximately 80 patients, with allowance of patients of 12 to 17 years of age who weigh less than 40 kg at the time of informed consent.

Patients enrolled in Cohort A receive emicizumab administration, with a loading dose of 3 mg/kg per administration once weekly (QW) for the first 4 weeks and a maintenance dose of 1.5 mg/kg per administration QW thereafter for a minimum of 52 weeks in total. Patients enrolled in Cohorts B and C receive emicizumab administration, with the same loading dose of 3 mg/kg QW for the first 4 weeks and a maintenance dose of 3 mg/kg Q2W (Cohort B) or 6 mg/kg Q4W (Cohort C) thereafter for a minimum of 52 weeks in total. During the 52-week treatment period, individual patients may have their dose up-titrated if they experience suboptimal bleeding control with emicizumab.

The efficacy analyses evaluate the clinical effect of prophylactic emicizumab on the number of bleeds over time (i.e., bleed rate), and characterize the efficacy of up-titration on an intra-patient level. Bleeds having different bleed definitions such as treated bleeds, all bleeds, treated spontaneous bleeds, treated joint bleeds, and treated target joint bleeds are analyzed separately. The primary analysis is performed 52 weeks after the last patient in the primary cohort, which consists of all patients enrolled in Cohort A prior to the close of enrollment for patients 2 years of age or older, has been enrolled or withdrawn prematurely, whichever occurs first. No formal hypothesis testing is planned in the study.

Results from an interim analysis in Cohort A are presented in Example 3.

### Example 7

A Multicenter, Open-Label, Phase III Study Evaluating the Efficacy, Safety, and Pharmacokinetics of Emicizumab Administered Every 2 or 4 Weeks in Pediatric Patients Less Than 12 Years Old with Hemophilia A without Factor VIII Inhibitors

This study is a multicenter, open-label, non-randomized study designed to evaluate the efficacy, safety, and pharmacokinetics of emicizumab administered subcutaneously at a dose of 3 mg/kg per administration once every 2 weeks (Q2W cohort) or 6 mg/kg per administration once every 4 weeks (Q4W cohort) in pediatric patients with hemophilia A without inhibitors. The study enrolls a minimum of 6 patients less than 12 years old with hemophilia A without inhibitors in each cohort.

The Q2W cohort of the study receives a loading dose of 3 mg/kg of emicizumab administered subcutaneously once weekly (QW) for the first 4 doses, followed by a maintenance dose of 3 mg/kg administered subcutaneously once every 2 weeks (Q2W) for at least 24 weeks in total. The Q4W cohort receives a loading dose of 3 mg/kg of emicizumab administered subcutaneously once weekly (QW) for the first 4 doses, followed by a maintenance dose of 6 mg/kg administered subcutaneously once every 4 weeks (Q4W) for at least 24 weeks in total. After Week 12 of emicizumab treatment, a higher dose may be selected for patients who meet the insufficient bleeding control criteria.

The efficacy analyses evaluate the clinical effects of emicizumab prophylaxis based on the bleeding frequency (bleeding rate) during a specified period. Bleeds having various different definitions such as bleeds requiring use of coagulation factors for hemostasis, spontaneous bleeds, joint bleeds, target joint bleeds, and all bleeds are analyzed separately. The primary efficacy analysis is conducted in each cohort when all patients have completed the 24-week treatment or have been withdrawn from the study, whichever occurs first. No statistical hypothesis testing is planned.

Sequence Listing

1. A method for treating and/or reducing the incidence of a disease that develops and/or progresses due to a decrease or deficiency in the activity of blood coagulation factor VIII and/or activated blood coagulation factor VIII (FVIIIa), the method comprising: administering to a subject a bispecific antibody that recognizes (a) blood coagulation factor IX and/or activated blood coagulation factor IX and (b) blood coagulation factor X and/or activated blood coagulation factor X at a weekly loading dose of 3 mg/kg or 4.5 mg/kg of the antibody for one or more weeks or at an every-two-week loading dose of 6 mg/kg of the antibody for two or more weeks; and, after the loading dose administration(s) are complete, administering a maintenance dose of the antibody to the subject one or more times, the maintenance dose being 6 mg/kg of the antibody.

2. The method of claim 1, wherein the maintenance dose of 6 mg/kg of the antibody is administered to the subject every four weeks or every month in a single dose or multiple divided doses.

3. The method of claim 1 or 2, wherein the antibody is administered at the weekly loading dose of 3 mg/kg of the antibody for four weeks, followed by the maintenance dose.

4. The method of claim 1 or 2, wherein the antibody is administered at the weekly loading dose of 4.5 mg/kg of the antibody for two weeks, followed by the maintenance dose.

5. The method of claim 1 or 2, wherein the antibody is administered at the every-two-week loading dose of 6 mg/kg of the antibody for four weeks, followed by the maintenance dose.

6. The method of any one of claims 1 to 5, wherein the maintenance dose is administered in one single dose at 6 mg/kg of the antibody monthly or every four weeks.

7. The method of any one of claims 1 to 5, wherein the maintenance dose is administered in two single doses of the antibody, each at 3 mg/kg, monthly or every four weeks, wherein one single dose of the maintenance dose at 3 mg/kg of the antibody is administered once every two weeks.

8. The method of any one of claims 1 to 5, wherein the maintenance dose is administered in four single doses each at 1.5 mg/kg of the antibody monthly or every four weeks, wherein one single dose of the maintenance dose at 1.5 mg/kg of the antibody is administered once every week.

9. The method of any one of claims 1 to 8 further comprising, in a case of no or insufficient effect of treating and/or reducing the incidence of the disease by administering the maintenance dose of the antibody, stopping administering the maintenance dose of the antibody and starting administering an alternative maintenance dose of the antibody to the subject, the alternative maintenance dose being a weekly dose of 3 mg/kg of the antibody or an every-two-week dose of 6 mg/kg of the antibody.

10. A method for treating and/or reducing the incidence of a disease that develops and/or progresses due to a decrease or deficiency in the activity of blood coagulation factor VIII and/or activated blood coagulation factor VIII (FVIIIa), the method comprising: administering to a subject a bispecific antibody that recognizes (a) blood coagulation factor IX and/or activated blood coagulation factor IX and (b) blood coagulation factor X and/or activated blood coagulation factor X at a weekly loading dose of 4.5 mg/kg of the antibody for four weeks; and thereafter administering a maintenance dose of the antibody to the subject one or more times, the maintenance dose being 9 mg/kg of the antibody which is administered every four weeks or every month in two or four divided doses.

11. A method for treating and/or reducing the incidence of a disease that develops and/or progresses due to a decrease or deficiency in the activity of blood coagulation factor VIII and/or activated blood coagulation factor VIII (FVIIIa), the method comprising: administering to a subject a bispecific antibody that recognizes (a) blood coagulation factor IX and/or activated blood coagulation factor IX and (b) blood coagulation factor X and/or activated blood coagulation factor X at a weekly loading dose of 6 mg/kg of the antibody for four weeks; and thereafter administering a maintenance dose of the antibody to the subject one or more times, the maintenance dose being 12 mg/kg of the antibody which is administered every four weeks or every month in two or four divided doses.

12. The method of claim 10 further comprising, in a case of no or insufficient effect of treating and/or reducing the incidence of the disease by administering the maintenance dose of the antibody, stopping administering the maintenance dose of the antibody and starting administering an alternative maintenance dose of the antibody to the subject, the alternative maintenance dose being a weekly dose of 4.5 mg/kg of the antibody.

13. The method of claim 11 further comprising, in a case of no or insufficient effect of treating and/or reducing the incidence of the disease by administering the maintenance dose of the antibody, stopping administering the maintenance dose of the antibody and starting administering an alternative maintenance dose of the antibody to the subject, the alternative maintenance dose being a weekly dose of 6 mg/kg of the antibody.

14. The method of any one of claims 1 to 13, wherein the antibody is emicizumab.

15. The method of claim 14, wherein the disease that develops and/or progresses due to a decrease or deficiency in the activity of blood coagulation factor VIII and/or activated blood coagulation factor VIII is selected from the group consisting of hemophilia A, acquired hemophilia A, von Willebrand disease, and hemophilia A with emergence of an inhibitor against blood coagulation factor VIII and/or activated blood coagulation factor VIII.


SEQUENCE LISTING (15 sequences)

SEQ ID NO: 1 - protein, 5 aa, synthetic construct
  REGION 1..5: note = Heavy chain variable region CDR1
YYDIQ

SEQ ID NO: 2 - protein, 17 aa, synthetic construct
  REGION 1..17: note = Heavy chain variable region CDR2
SISPSGQSTYYRREVKG

SEQ ID NO: 3 - protein, 14 aa, synthetic construct
  REGION 1..14: note = Heavy chain variable region CDR3
RTGREYGGGWYFDY

SEQ ID NO: 4 - protein, 123 aa, synthetic construct
  REGION 1..123: note = Heavy chain variable region
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSS

SEQ ID NO: 5 - protein, 448 aa, synthetic construct
  REGION 1..448: note = Heavy chain
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSKLTVDKSRWQEGNVFSCSVMHEALHNRYTQKSLSLSP

SEQ ID NO: 6 - protein, 5 aa, synthetic construct
  REGION 1..5: note = Heavy chain variable region CDR1
DNNMD

SEQ ID NO: 7 - protein, 17 aa, synthetic construct
  REGION 1..17: note = Heavy chain variable region CDR2
DINTRSGGSIYNEEFQD

SEQ ID NO: 8 - protein, 10 aa, synthetic construct
  REGION 1..10: note = Heavy chain variable region CDR3
RKSYGYYLDE

SEQ ID NO: 9 - protein, 119 aa, synthetic construct
  REGION 1..119: note = Heavy chain variable region
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSS

SEQ ID NO: 10 - protein, 444 aa, synthetic construct
  REGION 1..444: note = Heavy chain
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSP

SEQ ID NO: 11 - protein, 11 aa, synthetic construct
  REGION 1..11: note = Light chain variable region CDR1
KASRNIERQLA

SEQ ID NO: 12 - protein, 7 aa, synthetic construct
  REGION 1..7: note = Light chain variable region CDR2
QASRKES

SEQ ID NO: 13 - protein, 9 aa, synthetic construct
  REGION 1..9: note = Light chain variable region CDR3
QQYSDPPLT

SEQ ID NO: 14 - protein, 107 aa, synthetic construct
  REGION 1..107: note = Light chain variable region
DIQMTQSPSSLSASVGDRVTITCKASRNIERQLAWYQQKPGQAPELLIYQASRKESGVPDRFSGSRYGTDFTLTISSLQPEDIATYYCQQYSDPPLTFGGGTKVEIK

SEQ ID NO: 15 - protein, 214 aa, synthetic construct
  REGION 1..214: note = Light chain
DIQMTQSPSSLSASVGDRVTITCKASRNIERQLAWYQQKPGQAPELLIYQASRKESGVPDRFSGSRYGTDFTLTISSLQPEDIATYYCQQYSDPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC